(19) United States
(12) Patent Application Publication
(10) Pub. No.: US 2024/0009442 A1
(43) Pub. Date: Jan. 11, 2024

(54) PERCUTANEOUS ASSEMBLY AND METHODS OF USING SAME

(71) Applicant: Puzzle Medical Devices Inc., Montreal (CA)

(72) Inventors: Gabriel GEORGES, Sherbrooke (CA); Philippe GENEREUX, Basking Ridge, NJ (US)

(21) Appl. No.: 18/346,415

(22) Filed: Jul. 3, 2023

Related U.S. Application Data
(60) Provisional application No. 63/358,375, filed on Jul. 5, 2022.

(51) Int. Cl. A61M 60/13; A61M 60/295; A61M 60/497; A61M 60/865; A61F 2/24

(52) U.S. Cl. CPC A61M 60/13; A61M 60/295; A61M 60/497; A61M 60/865; A61F 2/2436

(57) ABSTRACT

A percutaneous assembly is provided with a percutaneous sheath and a percutaneous dilator tip. The percutaneous sheath has a tubular wall extending to a distal end portion thereof and defines a lumen having an inner diameter. The percutaneous dilator tip includes a radially expandable member and an elongated control member. The radially expandable member is either removably engageable or fixedly attached to the distal end portion of the tubular wall and is also adjustable in an expanded and a contracted configurations. The elongated control member is disposed longitudinally along the tubular wall, positioned eccentrically relative to the lumen, and is operatively coupled to the radially expandable member for adjusting same in at least one of the expanded and the contracted configurations. A percutaneous sheath is also provided as kits.

### CROSS-REFERENCE TO RELATED APPLICATION(S)

The present application claims priority to U.S. Provisional Patent Application No. 63/353,375, filed Jul. 5, 2022, the contents of which are incorporated herein by reference.

The present application is also related to International Patent Application Publication No. WO 2020/198,765 filed May 4, 2020, and entitled “Modular Mammalian Body Implantable Fluid Flow Influencing Device and Related Methods” (referred to hereinafter as the “WO '765 Publication”). The content of the 765 Publication is incorporated herein by reference in its entirety, including all references incorporated by reference therein, for all purposes as if fully set forth herein, except for any definitions, subject-matter disclaimers or disavowals, and except to the extent that the incorporated material is inconsistent with the express disclosure herein, in which case the language in the present disclosure controls.

### FIELD

The present disclosure generally relates to a percutaneous assembly and methods of using same.

### BACKGROUND

Minimally invasive medical procedures encompass surgical techniques that limit the size of incisions needed to conduct the procedures and so lessen wound healing time, associated pain, and risk of infection. Such procedures have been enabled by advances in various medical technologies, techniques, and instrumentations. Nowadays, minimally invasive medical procedures find applications in several medical specialties and sub-specialties, including cardiology, urology, nephrology, neurology, radiology, etc.

Transcatheter aortic valve replacement (TAVR) is an example of minimally invasive medical procedures in which a diseased aortic valve is replaced with a prosthetic aortic valve without involving invasive chirurgical procedures to open the chest in order to access the heart. Instead, access to the heart is generally obtained via a peripheral vessel, such as the femoral, subclavian, or axillary artery. Particularly, a needle is punctured through the skin and into the lumen of the blood vessel. A guidewire is passed through the needle and is guided along the blood vessel to the heart. Dilators of increasing diameters are sequentially railed over the guidewire and introduced into the puncture site in order to progressively increase the diameter thereof.

Upon reaching a puncture site of a sufficient diameter, a delivery sheath provided with a solid dilatator tip at an extremity thereof and containing a prosthetic aortic valve is railed over the guidewire and introduced with the solid dilator tip first through the puncture site. The delivery sheath is advanced along the vessel to an intraluminal site where the prosthetic aortic valve is to be deployed. Then, the solid dilator tip is removed away from the extremity of the delivery sheath by moving it further away along the vessel, and the prosthetic aortic valve is moved outside of the delivery sheath. In this case, since the prosthetic aortic valve is disposed between the delivery sheath and the solid dilator tip, the solid dilator tip needs to pass through the prosthetic aortic valve—e.g., though the opening(s) where the blood circulates upstream and downstream relative to the prosthetic aortic valve when deployed—in order to be put back in place at the extremity of the delivery sheath and/or to be removed from the vessel when the prosthetic aortic valve is deployed. Generally speaking, any other medical device, for example a stent, that is deployable inside a vessel using such a delivery sheath provided with a solid dilator tip also needs to enable passing a solid dilator tip therethrough for the same reasons.

Alternatively, an introducer sheath provided with a solid dilatator tip at an extremity thereof may be railed over a guidewire and introduced with the solid dilator tip first through a puncture site of a sufficient diameter. After removing the solid dilatator tip entirely from the introducer sheath by pulling it out therefrom, a delivery sheath (not provided with a dilatator tip) containing the prosthetic aortic valve may be railed over the guidewire, inserted inside the introducer sheath, and advanced along the vessel to a site where the prosthetic aortic valve is to be deployed. Then, the prosthetic aortic valve is moved outside of the delivery sheath, and the introducer and delivery sheaths are removed from the vessel. In this case, the insertion profile (reported in French size units) is increased as the delivery sheath is inserted inside the introducer sheath (that is, two nested sheaths are used). Such an increased insertion profile may limit or may even prevent the use of the introducer and delivery sheaths on vessels of smaller diameter.

The embodiments disclosed herein address these needs and other needs, as will become apparent from review of the specification hereinafter.

### SUMMARY

A first aspect on the present disclosure is directed to a percutaneous assembly, including:
a percutaneous sheath having a tubular wall extending to a distal end portion thereof, the tubular wall defining a lumen having an inner diameter;
a percutaneous dilator tip including a radially expandable member and an elongated control member;
   the radially expandable member having an expandable end portion removably engageable to the distal end portion of the tubular wall, the radially expandable member being adjustable in an expanded configuration in which the expandable end portion is configured to conform to the distal end portion of the tubular wall when engaged thereto, and a contracted configuration in which the expandable end portion has a contracted outer diameter that is smaller than the inner diameter of the lumen of the tubular wall; and
   the elongated control member being operatively coupled to the radially expandable member for adjusting the radially expandable member in at least one of the expanded configuration and the contracted configuration, the elongated control member further being longitudinally movable relative to the tubular wall when the expandable end portion of the radially expandable member is in the contracted configuration, the elongated control member further being eccentrically positioned relative to the lumen of the tubular wall.

In an embodiment, the expandable end portion of the radially expandable member is removably engageable by being removably insertable in the lumen of the tubular wall at the distal end portion thereof.

In an embodiment, the distal end portion of the tubular wall is configured for fitting snuggly with the expandable end portion of the radially expandable member adjusted in the expanded configuration when inserted in the lumen of the tubular wall.

In an embodiment, the distal end portion of the tubular wall includes an intraluminal portion having an inner diameter that is smaller than the expanded outer diameter of the expandable end portion of the radially expandable member when inserted in the lumen of the tubular wall.

In an embodiment, the radially expandable member includes an abutment wall configured for abutting to the distal end portion of the tubular when the expandable end portion of the radially expandable member adjusted in the expanded configuration is inserted in the lumen of the tubular wall.

In an embodiment, the expandable end portion of the radially expandable member adjusted in the expanded configuration has an expanded outer diameter that is substantially equal to the inner diameter of the lumen of the tubular wall when the expandable end portion is connected thereto.

In an embodiment, the elongated control member is longitudinally slidably receivable in the lumen of the tubular wall.

In an embodiment, the percutaneous sheath includes a secondary tubular wall extending at least partially along the tubular wall and defining a secondary lumen, the elongated control member being longitudinally slidably receivable in the secondary lumen.

In an embodiment, the percutaneous assembly includes a catheter for guiding the percutaneous assembly over a guidewire along a body conduit of a subject, the catheter being positioned eccentrically relative to the lumen of the tubular wall along at least a portion thereof.

In an embodiment, the radially expandable member includes a tapered end portion longitudinally opposed to the expandable end portion.

In an embodiment, the tapered end portion of the radially expandable member includes a reinforced distalmost narrower end.

In an embodiment, the radially expandable member is a balloon member of a balloon catheter.

In an embodiment, the elongated control member is a hose member of the balloon catheter and is configured for adjusting the balloon member in the expanded configuration by inflation and in the contracted configuration by deflation.

In an embodiment, the percutaneous assembly includes at least one medical device contained in the lumen of the tubular wall, at least one medical device being movable longitudinally outwardly from the lumen, at the distal end portion of the tubular wall, when the expandable end portion of the radially expandable member is removed from the distal end portion of the tubular wall.

In an embodiment, at least one medical device and the tubular wall together define a first inner sheath space therebetween where the elongated control member passes therethrough.

In an embodiment, the contracted outer diameter of the radially expandable member is smaller than the inner sheath space.

In an embodiment, at least one medical device is free of an opening adapted for passing the elongated control member therethrough.

In an embodiment, at least one medical device includes a first medical device and a second medical device disposed proximally to the first medical device, the first medical device having a first control wire connected thereto, the first control wire disposed longitudinally in the lumen of the tubular wall and positioned eccentrically relative to the lumen, the second medical device and the tubular wall together defining a second inner sheath space therebetween where the first control wire passes therethrough.

In an embodiment, at least one medical device includes at least one heart pump.

In an embodiment, at least one medical device includes a plurality of modular heart pumps assemblable together into a modular pump assembly.

A second aspect on the present disclosure is directed to a percutaneous assembly, including:
a percutaneous sheath having a tubular wall extending to a distal end portion thereof, the tubular wall having a circumference and defining a lumen having an inner diameter;
a percutaneous dilator tip including a radially expandable member and an elongated control member;
   the radially expandable member having an expandable end portion fixedly attached on a portion of the circumference of the tubular wall at the distal end portion thereof, the radially expandable member being adjustable in an expanded configuration in which the expandable end portion is configured to conform to the distal end portion of the tubular wall, and a contracted configuration in which the expandable end portion has a contracted outer diameter that is smaller than the inner diameter of the lumen of the tubular wall; and
   the elongated control member being operatively coupled to the radially expandable member for adjusting the radially expandable member in at least one of the expanded configuration and the contracted configuration, the elongated control member further being longitudinally disposed relative to the tubular wall and eccentrically positioned relative to the lumen thereof.

In an embodiment, the expandable end portion of the radially expandable member is received in the lumen of the tubular wall at the distal end portion thereof.

In an embodiment, the radially expandable member includes an abutment wall configured for abutting to the distal end portion of the tubular wall when the radially expandable member is adjusted in the expanded configuration.

In an embodiment, the expandable end portion of the radially expandable member adjusted in the expanded configuration has an expanded outer diameter that is substantially equal to the inner diameter of the lumen of the tubular wall.

In an embodiment, the elongated control is structurally integrated to the tubular wall.

In an embodiment, the percutaneous assembly includes a catheter for guiding the percutaneous assembly over a guidewire along a body conduit of a subject, the catheter being positioned eccentrically relative to the lumen of the tubular wall along at least a portion thereof.

In an embodiment, the catheter is structurally integrated to the tubular wall.

In an embodiment, the radially expandable member includes a tapered end portion longitudinally opposed to the expandable end portion.

In an embodiment, the tapered end portion of the radially expandable member includes a reinforced distalmost narrower end.

In an embodiment, the radially expandable member is a balloon member of a balloon catheter.

In an embodiment, the elongated control member is a hose member of the balloon catheter and is configured for adjusting the balloon member in the expanded configuration by inflation and in the contracted configuration by deflation.

In an embodiment, the percutaneous assembly includes at least one medical device contained in the lumen of the tubular wall, the at least one medical device being movable longitudinally outwardly from the lumen, at the distal end portion of the tubular wall, when the radially expandable member is adjusted in the contracted configuration.

In an embodiment, the at least one medical device and the tubular wall together define a first inner sheath space therebetween where the elongated control member passes therethrough.

In an embodiment, the at least one medical device is free of an opening adapted for passing the elongated control member therethrough.

In an embodiment, the at least one medical device includes a first medical device and a second medical device disposed proximally to the first medical device, the first medical device having a first control wire connected thereto, the first control wire disposed longitudinally in the lumen of the tubular wall and positioned eccentrically relative to the lumen, the second medical device and the tubular wall together defining a second inner sheath space therebetween where the first control wire passes therethrough.

In an embodiment, the at least one medical device includes at least one heart pump.

In an embodiment, the at least one medical device includes a plurality of modular heart pumps assemblable together into a modular pump assembly.

A third aspect on the present disclosure is directed to a kit, including:
a) a percutaneous sheath having a tubular wall extending to a distal end portion thereof, the tubular wall defining a lumen having an inner diameter;
b) a percutaneous dilator tip including a radially expandable member and an elongated control member;
   the radially expandable member having an expandable end portion removably engageable to the distal end portion of the tubular wall, the radially expandable member being adjustable in an expanded configuration in which the expandable end portion is configured to conform to the distal end portion of the tubular wall when engaged thereto, and a contracted configuration in which the expandable end portion has a contracted outer diameter that is smaller than the inner diameter of the lumen of the tubular wall; and
   the elongated control member being operatively coupled to the radially expandable member for adjusting the radially expandable member in at least one of the expanded configuration and the contracted configuration, the elongated control member further being longitudinally movable relative to the tubular wall when the expandable end portion of the radially expandable member is in the contracted configuration, the elongated control member further being eccentrically positioned relative to the lumen of the tubular wall.

A fourth aspect on the present disclosure is directed to a method of deploying a medical device inside a body conduit of a subject through a body conduit access, the method including:
introducing a percutaneous assembly through the body conduit access inside the body conduit, the percutaneous assembly includes a percutaneous sheath having a tubular wall extending to a distal end portion thereof and defining a lumen, and a percutaneous dilator tip having a radially expandable member disposed at the distal end portion of the tubular wall and adjusted in an expanded configuration, the tubular wall containing the medical device in the lumen thereof;
moving the radially expandable member distally away from the distal end portion of the tubular wall;
moving the medical device outside of the lumen of the tubular wall inside the body conduit;
adjusting the radially expandable member in a contracted configuration; and
removing the percutaneous assembly from the body conduit through the body conduit access.

In an embodiment, the method includes: preparing a body conduit access to the body conduit.

In an embodiment, the introducing step includes: advancing the percutaneous assembly inside the body conduit.

In an embodiment, the introducing step includes: advancing the percutaneous assembly over a guidewire inside the body conduit.

In an embodiment, the removing step includes: passing the radially expandable member adjusted in the contracted configuration through an inner body conduit space defined by the medical device and the body conduit.

In an embodiment, the method includes: closing the body conduit access of the body conduit.

Features and advantages of the disclosed subject-matter will become apparent in view of the following detailed description of selected embodiments, as illustrated in the accompanying drawings.

### Definition(s)

As intended herein, the terms “longitudinal” and “longitudinally” refer to a reference axis and/or a reference plane running lengthwise relative to the element being disclosed herein.

The terms “transversal” and “transversally” refer to a reference axis and/or a reference plane running widthwise relative to the element being disclosed herein.

The terms “proximal” and “proximally” refer to a point that is close to the operator of the element being disclosed herein.

The terms “distal” and “distally” refer to a point that is distant from the operator of the element being disclosed herein.

The terms “concentric” and “concentrically” refer to a physical disposition, arrangement, or configuration correlating with the geometric center.

The terms “eccentric” and “eccentrically” refer to a physical disposition, arrangement, or configuration that is non-concentric and therefore that does not correlate with the geometric center.

The term “plurality” refers to “two or more”.

The terms “aspect”, “one aspect”, “an aspect”, “another aspect”, “the aspect”, “one or more aspects”, “some aspect(s)”, “certain aspect(s)” and the like are intended to mean “one or more (but not all) aspects of the disclosed subject-matter,” unless expressly specified otherwise. Similarly, the terms “embodiment”, “one embodiment”, “an embodiment”, “another embodiment”, “the embodiment”, “one or more embodiments”, “some embodiment(s)”, “certain embodiment(s)” and the like are intended to mean “one or more (but not all) embodiments of the disclosed subject-matter,” unless expressly specified otherwise. Moreover, reference to “another aspect” or “another embodiment” does not imply that the referenced aspect or referenced embodiment is mutually exclusive with any other aspect(s) or embodiment(s), unless expressly specified otherwise.

### BRIEF DESCRIPTION OF THE DRAWINGS

In order that the present disclosure be readily understood, at least some selected embodiments thereof are illustrated by way of example(s) in the accompanying drawings.

FIG. 1 is a perspective view of a percutaneous assembly with a radially expandable member adjusted in an expanded configuration, according to an aspect of the present disclosure.

FIG. 2A is a perspective view of the percutaneous assembly of FIG. 1 with a guidewire routed therethrough; the tubular wall of the percutaneous assembly being illustrated in transparency.

FIG. 2B is an enlarged fragmentary cross-section view of the percutaneous assembly of FIG. 1 with a guidewire routed therethrough.

FIG. 3A is a perspective view of the percutaneous assembly of FIG. 1 with the radially expandable member adjusted in a contracted configuration; the tubular wall of the percutaneous assembly being illustrated in transparency.

FIG. 3B is an enlarged fragmentary cross-section view of the percutaneous assembly of FIG. 1 with the radially expandable member adjusted in a contracted configuration.

FIG. 4 is an enlarged fragmentary cross-section view of a percutaneous assembly with a hose member and a radially expandable member being a balloon member adjusted in an expanded configuration, according to an embodiment of the present disclosure.

FIGS. 5A-B are enlarged fragmentary cross-section views of a percutaneous assembly with a hose member and a radially expandable member being another balloon member adjusted in an expanded configuration, according to an embodiment of the present disclosure.

FIG. 6 is an enlarged fragmentary cross-section view of the percutaneous assembly of FIG. 1 with the radially expandable member adjusted in the contracted configuration, and an elongated control member slidably received in a lumen of the tubular wall.

FIG. 7A is an enlarged fragmentary cross-section view of a percutaneous assembly with a secondary tubular wall defining a secondary lumen, and an elongated control member slidably received in the secondary lumen, according to an embodiment of the present disclosure.

FIG. 7B is a cross-section view of the percutaneous sheath of the percutaneous assembly of FIG. 7A.

FIG. 8 is an enlarged fragmentary cross-section view of a percutaneous assembly with the radially expandable member fixedly attached to the tubular wall of the percutaneous assembly, according to an embodiment of the present disclosure.

FIG. 9 is a perspective view of a percutaneous assembly with an elongated control member passing through an aperture of the tubular wall of the percutaneous assembly, according to an embodiment of the present disclosure.

FIG. 10A is an enlarged fragmentary cross-section view of a percutaneous assembly with a radially expandable member that is reinforced, according to an embodiment of the present disclosure.

FIG. 10B is an enlarged fragmentary cross-section view of a percutaneous assembly with a radially expandable member that is reinforced, and an elongated control member slidably received in a secondary lumen defined by a secondary tubular wall, according to an embodiment of the present disclosure.

FIG. 11 is an enlarged fragmentary cross-section view of a percutaneous assembly with a tubular wall having an intraluminal portion of a reduced inner diameter, according to an embodiment of the present disclosure.

FIG. 12 is an enlarged fragmentary cross-section view of a percutaneous assembly with a radially expandable member having an abutment wall, according to an embodiment of the present disclosure.

FIGS. 13A-B and 14A-B are enlarged fragmentary cut away views of the percutaneous assembly of FIG. 1 in various introduction and deployment arrangements, according to embodiments of the present disclosure.

FIGS. 15 and 16 are block diagrams representing various methods of deploying a medical device inside a body conduit of a subject using a percutaneous assembly, according to an aspect of the present disclosure.

It will be noted that like reference numerals identify similar parts and/or features throughout the several views of the drawings. Moreover, the features illustrated in the drawings are not necessarily drawn to scale.

### DETAILED DESCRIPTION

The subject matter of the present disclosure, along with any advantage thereof, is described and explained in the following detailed description with reference to the non-limiting aspect(s), embodiment(s), example(s), feature(s), element(s), and step(s), as the case may be, presented hereinafter and illustrated in the accompanying non-limiting drawings and/or figures. Recognizing that these non-limiting aspect(s), embodiment(s), example(s), feature(s), element(s), and step(s) may vary, the skilled addressee shall readily recognize that any other variants thereof and any combination of these other variants, as the case may be, are contemplated without departing from the scope of the present disclosure, even if they are all not explicitly presented and stated herein.

Therefore, these non-limiting aspect(s), embodiment(s), example(s), feature(s), and/or element(s) is/are intended merely to facilitate an understanding of ways in which the claimed subject matter may be reduced to practice by the skilled addressee. Accordingly, these non-limiting aspect(s), embodiment(s), example(s), feature(s), and/or element(s) shall not be construed as limiting the scope of the claimed subject matter, which is defined solely by the accompanying claims and applicable law.

The terminology used herein is only for the purpose of describing and explaining the claimed subject matter and is not intended to limit the scope hereof. Unless defined otherwise, all technical, engineering, scientific, and other relevant terminology used herein have the same meanings as commonly understood by the skilled addressee.

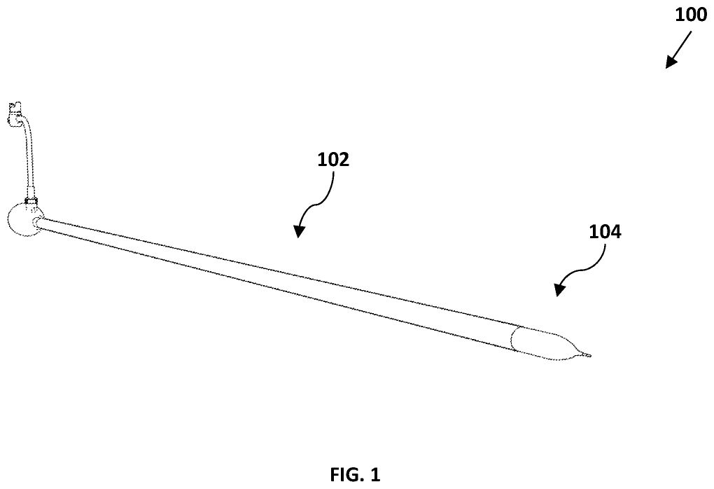

With respect to FIG. 1, there is disclosed a percutaneous assembly 100 designed to be introduced into a body conduit of a subject and provided with a percutaneous sheath 102 and a percutaneous dilator tip 104 that is removably engageable to the percutaneous sheath 102, in accordance with a first aspect of the present disclosure. In the context of cardiovascular application, the percutaneous assembly 100 is designed to be introduced in the lumen of blood vessels in order to provide medical practitioners with vascular access to perform various medical procedures. Depending on the medical procedure to be performed, the percutaneous assembly 100 may be of various length, cross-section size, and cross-section shape in order to provide vascular accesses of different natures. Particularly, as it will become apparent hereinafter, the percutaneous assembly 100 is useful for deploying at least one medical device, such as at least one heart pump, in the lumen of the vasculature in order to provide hemodynamic support to the heart.

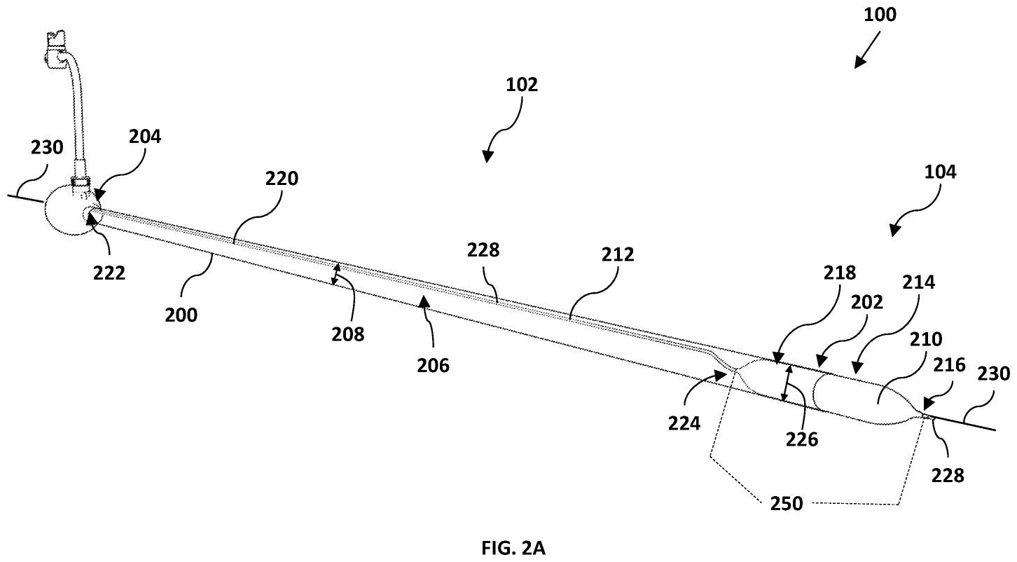

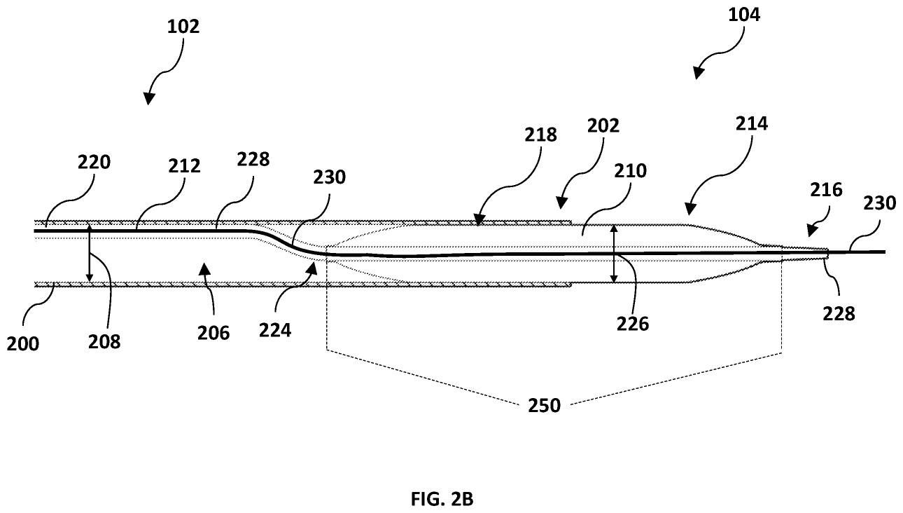

FIGS. 2A-B illustrate the percutaneous assembly 100 provided with the percutaneous sheath 102 and the percutaneous dilator tip 104 that is at least partially received inside the percutaneous sheath 102, according to an embodiment. The percutaneous sheath 102 is of a tubular hollow shape and has a tubular wall 200 extending between a distal end portion 202 and a proximal end portion 204 thereof. The tubular wall 200 defines a lumen 206 having an inner diameter 208 and extending longitudinally between the distal and proximal ends 202, 204.

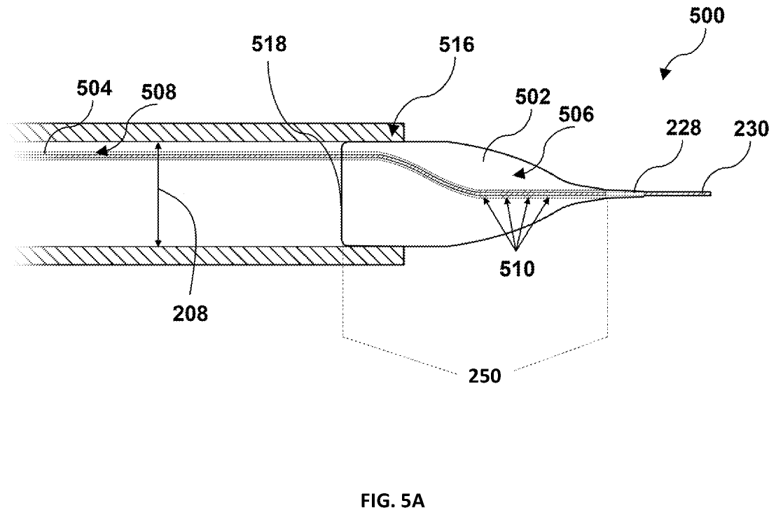

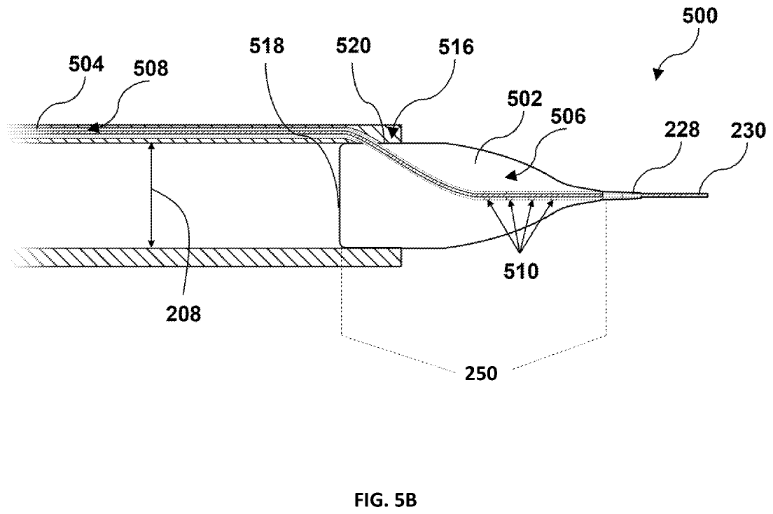

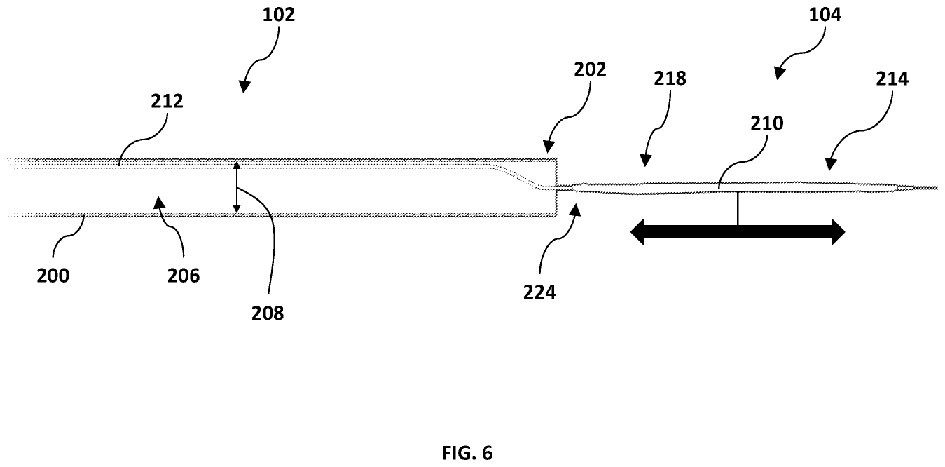

The percutaneous dilator tip 104 is provided with a radially expandable member 210 and an elongated control member 212. The radially expandable member 210, which is radially expandable and radially contractable, is of a circular transversal cross-section shape, and has a tapered end portion 214 having a distalmost narrower end 216 and an expandable end portion 218 that is longitudinally opposed to the tapered end portion 214. In an embodiment, as illustrated in FIGS. 2A-B, the expandable end portion 218 has a tapered shape generally similar to the tapered end portion 214 (i.e., the radially expandable member 210 is of a symmetrically longitudinal tapered shape), while in another embodiment the expandable end portion 218 has a transversal flat shape having a flat end wall (not shown) that is generally similar to the flat end wall 518 illustrated in FIGS. 5A-B.

The elongated control member 212 has an elongated body 220 extending between a proximal end portion 222 and a distal end portion 224 thereof. As illustrated, the expandable end portion 218 of the radially expandable member 210 is inserted at least partially in the lumen 206 of the tubular wall 200, while the elongated body 220 is disposed longitudinally in the lumen 206 of the tubular wall 200 and is positioned eccentrically relative to the lumen 206 thereof. The distal end portion 224 of the elongated body 220 is operatively coupled to the expandable end portion 218 for adjusting the radially expandable member 210 in at least one of an expanded configuration 250 and a contracted configuration 350, as it will become apparent hereinafter (the radially expandable member 210 is illustrated in the expanded configuration 250 in FIGS. 2A-B).

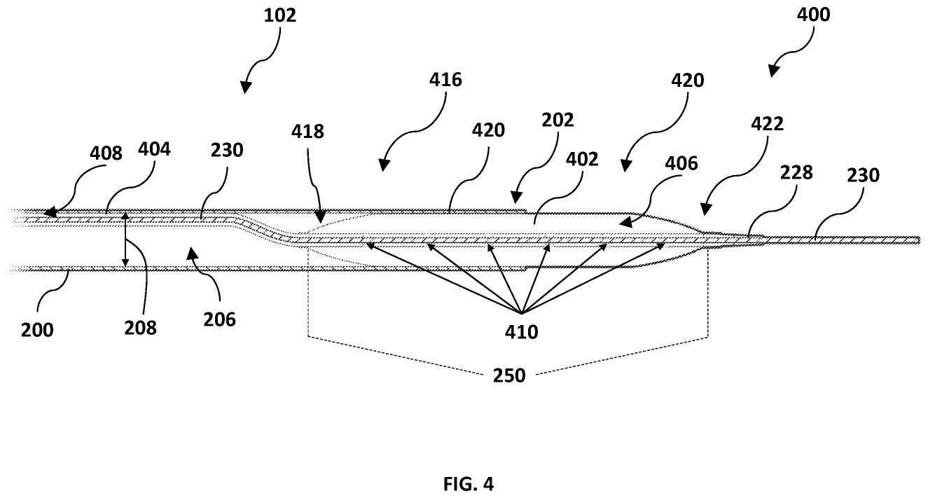

In embodiments, as illustrated in FIGS. 2A-B and also in FIGS. 4, 5A-B, 9, 10A-B, 11, 13A-B, and 14A-B, the percutaneous assembly 100 may further be provided with a catheter 228 and a guidewire 230 slidably received in the catheter for guiding the percutaneous assembly 100 along a body conduit of a subject, as known in the art. As illustrated, the catheter is structurally integrated to the elongated control member 212 and runs between the proximal end portion 222 thereof and the distalmost narrower end 216 of the radially expandable member 210. Particularly, the catheter 228 and the guidewire 230, if present, are disposed longitudinally relative to percutaneous sheath 102 and are positioned eccentrically relative to the lumen 206 of the tubular wall 200.

In other embodiments, the catheter 228 may be provided to the percutaneous assembly 100 without necessarily being structurally integrated to the elongated control member 212. For example, the catheter 228 may run between the proximal end portion 222 of the tubular wall 200 and the distalmost narrower end 216 of the radially expandable member 210 as a separate component from the elongated control member 212.

Still in other embodiments, the catheter 228 may be structurally integrated to the tubular wall 200, and therefore physically contained within the thickness thereof, independently of and as a separate component from the elongated control member 212.

For the sake of brevity, only the distal end portion 202 of the tubular wall 200 and the distal end portion 224 of the elongated control member 212 will be described hereinafter since the proximal end portion 204 of the tubular wall 200 and the proximal end portion 222 of the elongated control member 212 will become apparent to the skilled addressee as being the proximal manipulation end of the operator.

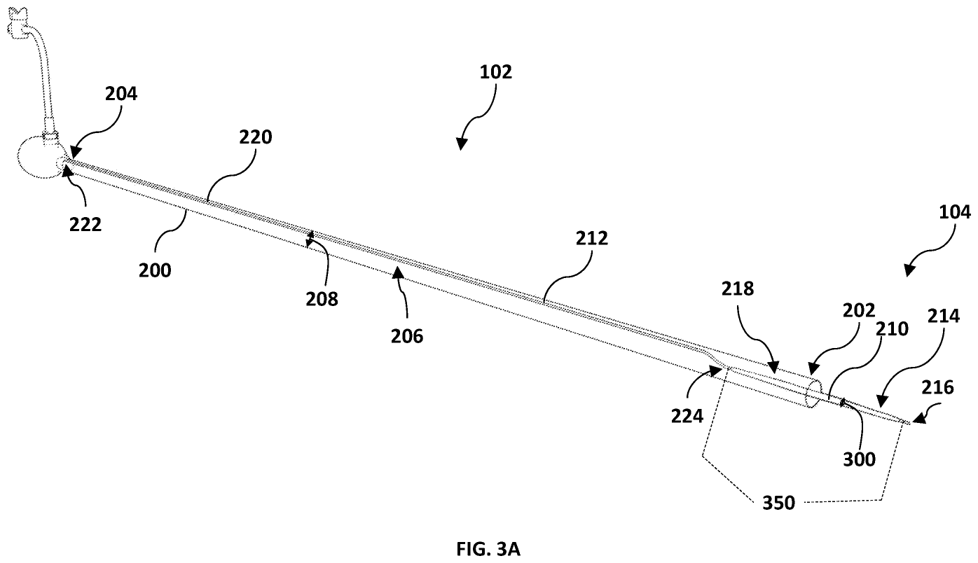

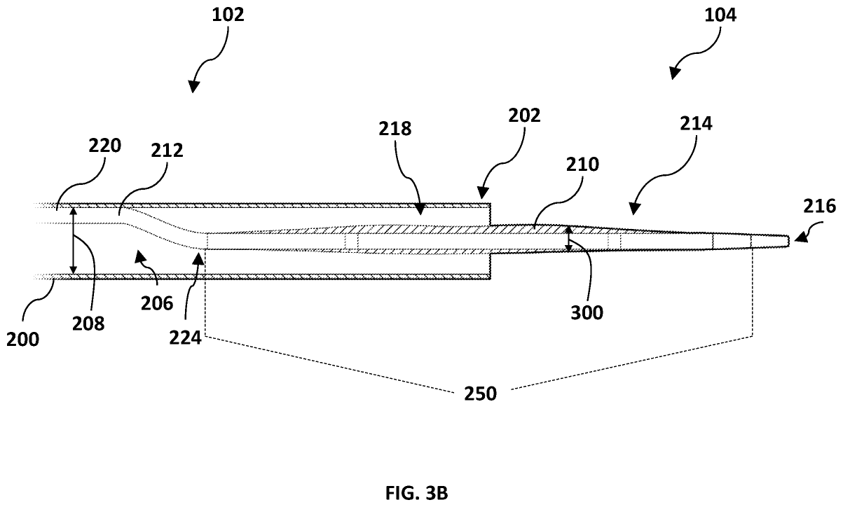

Still referring to FIGS. 2A-B, and also to FIGS. 3A-B, the radially expandable member 210 of the percutaneous dilator tip 104 is adjustable in the expanded configuration 250 (FIGS. 2A-B) and the contracted configuration 350 (FIGS. 3A-B). As illustrated, the radially expandable member 210 is inserted at least partially inside the percutaneous sheath 102 at the distal end position 202 thereof and is operatively coupled to the elongated control member 212 for adjusting the radially expandable member 210 in the expanded and contracted configurations 250, 350.

In FIGS. 2A-B, the radially expandable member 210 is adjusted in the expanded configuration 250 and has the expandable end portion 218 thereof inserted in the lumen 206 of the tubular wall 200. As such, when the radially expandable member 210 is adjusted in the expanded conformation 250, the expandable end portion 218 structurally conforms in size and shape with the lumen 206 of the tubular wall 200. So inserted, the expandable end portion 218 has an expanded outer diameter 226 that is equal or substantially equal to the inner diameter 208 of the lumen 206. As illustrated, the expanded outer diameter 226 is so sized that there is no significant circumferential gap between the expandable end portion 218 of the radially expandable member 210 and the tubular wall 200 of the percutaneous sheath 102. When present, such significant circumferential gap increases the risk that the distal end portion 202 of the tubular wall 200 scratches and/or damages the endoluminal surface of the body conduit, such as the intima on the case of blood vessel, into which the percutaneous assembly 100 is being introduced. This is because a significant circumferential gap disrupts an otherwise smooth, progressive transition between the tapered end portion 214 of the radially expandable member 210 and the distal end portion 202 of the tubular wall 200 where the radially expandable member 210 is present. Such scratching and/or damaging of the endoluminal surface is exacerbated when the distal end 202 of the tubular wall 200 has a sharp edge.

The expandable end portion 218 of the radially expandable member 210 adjusted in the expanded configuration 250 may fit snuggly to the distal end portion 202 of the tubular wall 200 while inserted in the lumen 206 thereof. The snug fit may result in a frictional engagement between the expandable end portion 218 and the tubular wall 200 that prevents the radially expandable member 210 from being displaced in the lumen 206 of the tubular wall 200 while a longitudinal force is applied (e.g., by an operator) to the tapered end portion 214 during introduction of the percutaneous assembly 100 inside a body conduit. The snug fit may also result in a fluid-tight engagement (i.e., a sealed connection) between the expandable end portion 218 and the tubular wall 200 that prevents fluid, such as blood and other biological fluids, from penetrating in the lumen 206 of the tubular wall 200, thereby protecting the internal component(s) and/or content (e.g., heart pump(s), when present) of the percutaneous assembly 100.

The skilled addressee will appreciate that the expandable end portion 218 of the radially expandable member 210 may have an expanded outer diameter 226 that is larger than the inner diameter 208 of the lumen 206 when the radially expandable member 210 is adjusted in the expanded configuration 250 outside of the lumen 206, but yet that the expandable end portion 218 in fact has an expanded outer diameter 226 that is equal or substantially equal to the inner diameter 208 of the lumen 206 when the radially expandable member 210 is adjusted in the expanded configuration 250 in the lumen 206. This may be the case, for example, when the expandable end portion 218 of the radially expandable member 210 intrinsically has an expanded outer diameter 226 that is larger than the inner diameter 208 of the lumen 206 of the tubular wall 200.

Referring now to FIGS. 3A-B, the radially expandable member 210 adjusted in the contracted configuration 350 has the expandable end portion 218 thereof inserted in the lumen 206 of the tubular wall 200. So inserted, the expandable end portion 218 has a contracted outer diameter 300 that is smaller than the inner diameter 208 of the lumen 206 of the tubular wall 200. As such, the contracted outer diameter 300 is so sized that there is a circumferential gap between the expandable end portion 218 of the radially expandable member 210 and the tubular wall 200 of the percutaneous sheath 102. As it will become apparent hereinafter, the adjustment of the radially expandable member 210 in the contracted configuration 350 not only enables the percutaneous assembly 100 to be removed from the body conduit where it has been introduced after deployment of a medical device, but also enables the radially expandable member 210 to be moved towards or even inside the tubular wall 200 in presence or absence of a medical device contained therein.

While the distal end portion 224 of the elongated control member 212 is operatively coupled to the expandable end portion 218 of the radially expandable member 210 in the illustrated embodiments, the distal end portion 224 of the elongated control member 212 may be operatively coupled to the tapered end portion 214 of the radially expandable member 210 in other embodiments (not shown). Furthermore, while the radially expandable member 210 is provided with only one elongated control member 212 in the illustrated embodiments, two elongated control members 212 may also be provided with the radially expandable member 210 in other embodiments (not shown). In this case, each of the two elongated control members 212 is operatively coupled to the radially expandable member 210. One elongated control member 212 is configured for adjusting the radially expandable member 210 in the expanded configuration 250, and the other elongated control member 212 is configured for adjusting the radially expandable member 210 in the contracted configuration 350.

In a preferred embodiment, illustrated in FIG. 4, the percutaneous dilator tip 104 is a balloon catheter 400, the radially expandable member 210 is a balloon member 402 of the balloon catheter 400, and the elongated control member 212 is a hose member 404 of the balloon catheter 400. The balloon member 402 has a fluid-fillable reservoir 406 that is in fluid communication with a fluid passageway 408 of the hose member 404 via a plurality of fluid ports 410. The balloon member 402 has a proximal tapered end portion 416 (which correspond to the expandable end portion 218 of the radially expandable member 210) having a proximal narrower end 418 and a distal tapered end portion 420 (which correspond to the tapered end portion 214) having a distal narrower end 422. The hose member 404 is operatively coupled to the proximal narrower end 418 of the balloon member 402 such that the fluid passageway 408 is in fluid communication with the fluid-fillable reservoir 406. Alternatively, the hose member 404 may be operatively and eccentrically coupled to a circumferential area 420 of the proximal tapered end portion 416 of the balloon member 402 (this connection is not shown). In an embodiment, as illustrated, the balloon catheter 400 may also be provided with a guidewire 230 slidably received through a lumen running along the balloon member 402 and the hose member 404 which may be used for guiding the percutaneous assembly along a body conduit of a subject into which the percutaneous assembly 100 is advanced, as known in the art.

The balloon member 402 may be adjusted in the expanded configuration 250 (as illustrated in FIG. 4) by inflating the fluid-fillable reservoir 406 with a fluid (resulting in the radial expansion of the balloon member 402), and to the contracted configuration 350 by deflating the fluid-fillable reservoir 406 from a fluid (resulting in the radial contraction of the balloon member 402). The circulation of the fluid in the fluid passageway 408 of the hose member 404 to and from the fluid-fillable reservoir 406 to inflate and deflate the balloon member 402 may be performed by a pump and/or any other fluid circulating apparatus know in the art (not shown), which is/are in fluid communication with the fluid passageway 408. For example, the pump and/or fluid circulating apparatus may be operatively coupled to a proximal end portion (not shown) of the hose member 402.

In other preferred embodiments, illustrated in FIGS. 5A-B, the percutaneous dilator tip 104 is a balloon catheter 500, the radially expandable member 210 is a balloon member 502 of the balloon catheter 500, and the elongated control member 212 is a hose member 504 of the balloon catheter 500. The balloon member 502 has a fluid-fillable reservoir 506 that is in fluid communication with a fluid passageway 508 of the hose member 504 via a plurality of fluid ports 510. In an embodiment, as illustrated, the balloon catheter 500 may also be provided with a guidewire 230 slidably received through a lumen running along the balloon member 502 and the hose member 504 which may be used for guiding the percutaneous assembly along a body conduit of a subject into which the percutaneous assembly 100 is advanced, as known in the art.

The preferred embodiments illustrated in FIGS. 5A-B are therefore generally similar to the one illustrated in FIG. 4 but differs in that the balloon member 502 has a proximal end portion 516 having a flat end wall 518. In the embodiment illustrated in FIG. 5A, the hose member 504 is disposed in the lumen 206 of the tubular wall 200 and is operatively and eccentrically coupled to the flat end wall 518, for example about the circumference thereof, such that the fluid passageway 508 is in fluid communication with the fluid-fillable reservoir 506. In the embodiment illustrated in FIG. 5B, the hose member 504 is structurally integrated to the tubular wall 200, and therefore physically contained within the thickness thereof, and is operatively and eccentrically coupled to a circumferential area 520 of the proximal end portion 516 of the balloon member 502. It is contemplated that any radially expandable member 210—not only the balloon member 502—may be provided with a flat end wall 518, and that any elongated control member 212—not only the hose member 504, may be operatively coupled to the radially expandable member 210 via the flat end wall 518 or the circumferential area 520.

In embodiments, the radially expandable member 210 of the percutaneous dilator tip 104 is removably engageable to the percutaneous sheath 102. Therefore, as it will become apparent hereinafter, the elongated control member 212 serves not only to adjust the radially expandable member 210 in the expanded and contracted configurations 400, 450, but also serve to move longitudinally outwardly and inwardly the radially expandable member 210 relative to the tubular wall 200.

In an embodiment, illustrated in FIG. 6, the radially expandable member 210 is removably engageable to the tubular wall 200 by being removably insertable at least partially in the lumen 206 of the tubular wall 200, at the corresponding distal end portion 202 thereof (this is indicated by the bold double-headed arrow in FIG. 6). As illustrated, the radially expandable member 210 is adjusted in the contracted configuration 350 but may also be adjusted in the expanded configuration 250 while still being removably engageable (although, depending on embodiment(s), the radial size of the radially expandable member 210 may be required to be somewhat reduced to facilitate a removable engagement). The elongated control member 212 of the percutaneous dilator tip 104 is in this case slidably received in the lumen 206 of the tubular wall 200 of the percutaneous sheath 102. When the radially expandable member 210 is inserted in the lumen 206, the elongated control member 212 extends longitudinally between the distal and proximal end portions 202, 204 of the tubular wall 200 and is positioned eccentrically relative to the lumen 206 thereof. The radially expandable member 210 of the percutaneous dilator tip 104 may be removed from the percutaneous sheath 102 by applying a pushing force in order to slide longitudinally the elongated control member 212 outwardly in direction of the distal end portion 202 of the tubular wall 200. On the other hand, the radially expandable member 210 of the percutaneous dilator tip 104 may be inserted inside the percutaneous sheath 102 by applying a pulling force in order to slide longitudinally the elongated control member 212 inwardly in direction of the proximal end portion 204 of the tubular wall 200. For example, an operator may apply the pushing force and the pulling force to the proximal end portion 222 of the elongated control member 212 to remove the radially expandable member 210 from the lumen 206 of the tubular wall 200 and to insert the radially expandable member 210 into the lumen 206 of the tubular wall 200, respectively.

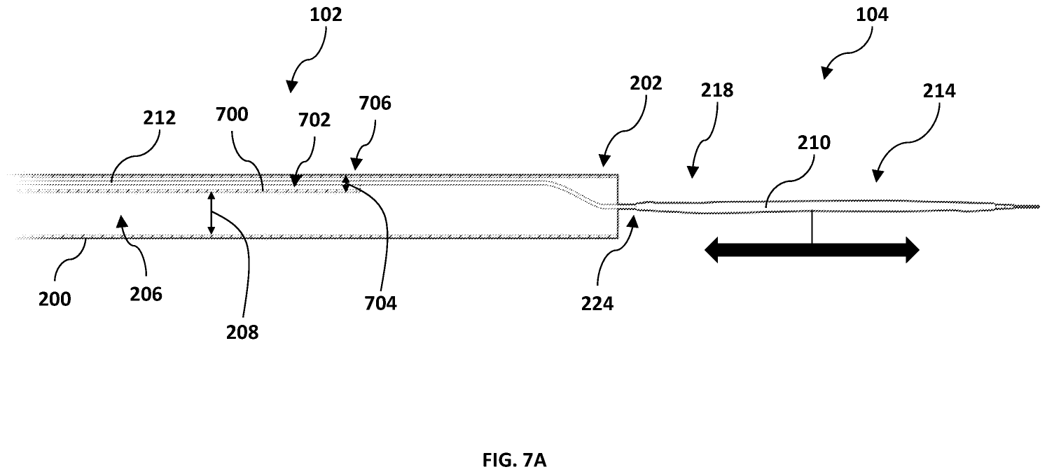

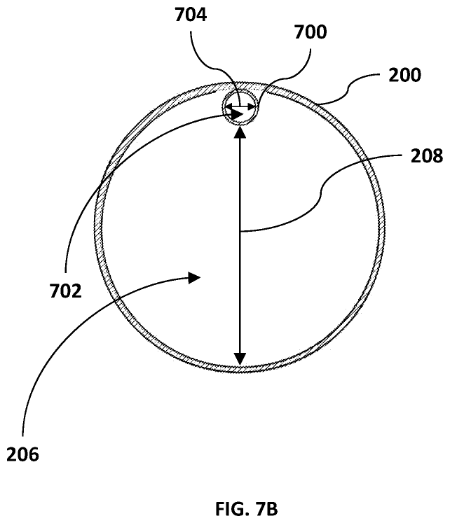

In another embodiment, illustrated in FIGS. 7A-B, the percutaneous sheath 102 is further provided with a secondary tubular wall 700 defining a secondary lumen 702 having a secondary inner diameter 704 (best shown in FIG. 7B). As described before, the radially expandable member 210 is removably engageable to the tubular wall 200 by being removably insertable at least partially in the lumen 206 of the tubular wall 200, at the corresponding distal end portion 202 thereof (this is indicated by the bold double-headed arrow in FIG. 7A). However, the elongated control member 212 of the percutaneous dilator tip 104 is, in this case, slidably received in the secondary lumen 702 of the secondary tubular wall 700. As illustrated in FIG. 7A, the secondary tubular wall 700 is positioned eccentrically relative to the lumen 206 of the tubular wall 200. The secondary tubular wall 700 has a proximal end portion (not shown) and a distal end portion 706 and extends at least partially between the corresponding distal and proximal end portions 202, 204 of the tubular wall 200. The sliding action of the elongated control member 212 inside the secondary lumen 702 to move outwardly and inwardly the radially expandable member 210 relative to the lumen 206 of the tubular wall 200 is as described for the elongated control member 212 slidably received in the lumen 206 of the tubular wall 200 hereinbefore.

Advantageously, the secondary tubular wall 700 forms a pathway that physically isolates the elongated control member 212 from the other component(s) and/or the content of the tubular wall 200, thereby preventing those internal component(s) and/or content from interfering with the functioning of the elongated control member 212. This may be the case, for example, in embodiments where the elongated control member 212 is a hose member 404, 504, which may be collapsed due to the presence of a medical device contained in the lumen 206 of the tubular wall 200.

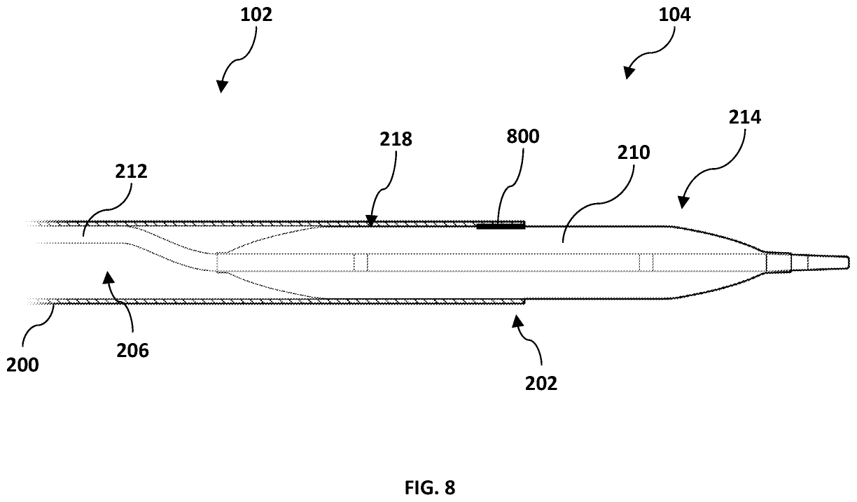

In embodiments, the percutaneous dilator tip 104 is fixedly attached to the percutaneous sheath 102. Particularly, as illustrated in the embodiments of FIG. 8, the radially expandable member 210 of the percutaneous dilator tip 104 is fixedly attached to the tubular wall 200 of the percutaneous sheath 102 such that the elongated control member 212 cannot slide longitudinally inside the percutaneous sheath 102. The expandable end portion 218 of the radially expandable member 210 is received in the lumen 206 of the tubular wall 200, at the distal end portion 202 thereof, and is fixedly attached in the lumen 206 by an attachment point 800 that runs along a portion of the circumference of the tubular wall 200. The attachment point 800 runs partially along the circumference of the tubular wall 200 so as to provide sufficient attachment strength during use between the radially expandable member 210 and the tubular wall 200, while also providing sufficient luminal clearance at the distal end portion 202 of the tubular wall 200 so that medical device(s) contained in the lumen 206 may still be moved outside therefrom when the radially expandable members is in the contracted configuration 350, as it will become apparent hereinafter. For example, the attachment point may run about a quarter (⅓), a third (¼), a fifth (⅕), a sixth (⅙), a seventh ( 1/7), an eighth (⅛), a nineth ( 1/9), a tenth ( 1/10), an eleventh ( 1/11), or a twelfth ( 1/12) of the circumference of the tubular wall 200, at the distal end portion 204 thereof. Exemplary fixed attachments of the expandable end portion 218 of the radially expandable member 210 include fusing, welding, gluing, etc.

While in the embodiment illustrated in FIG. 8 the elongated control member 212 is received in the lumen 206 of the tubular wall 200, in other embodiments the elongated control member 212 may be received in the secondary lumen 702 of the secondary tubular wall 700 (e.g., as illustrated in the embodiment of FIG. 7A) or be structurally integrated to the tubular wall 200 and therefore physically contained within the thickness thereof (e.g., as illustrated in the embodiment of FIG. 5B, where an attachment point 800 (not shown) located at the circumferential area 520 may fixedly attached the radially expandable member 210 to the tubular wall 200).

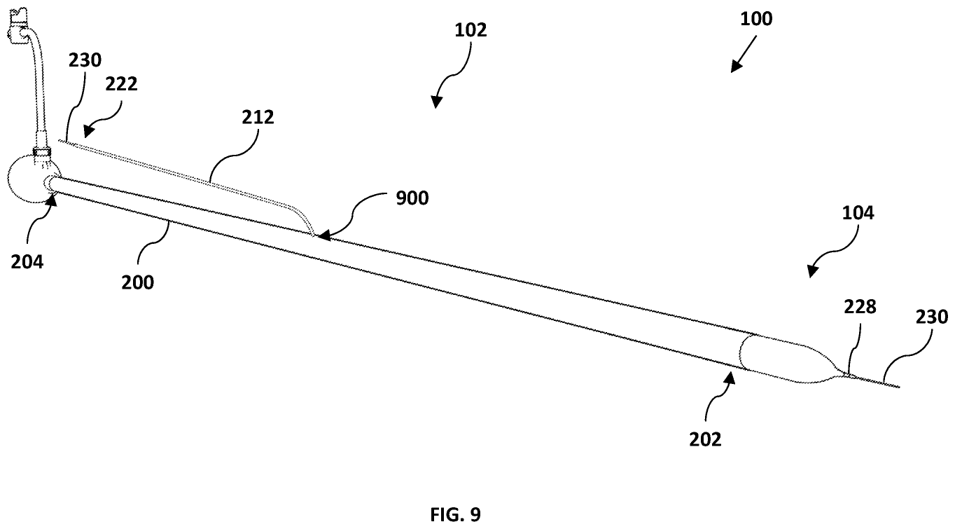

FIG. 9 illustrates an embodiment of the percutaneous assembly 100 provided with a percutaneous sheath 102 having an aperture 900 in the tubular wall 200 for receiving the elongated control member 212 therethrough. When the elongated control member 212 is received though the aperture 900, the proximal end portion 222 of the elongated control member 212 is at least partially disposed longitudinally outside of the tubular wall 200, towards the proximal end portion 204 thereof, and the distal end portion 224 of the elongated control member 212 is at least partially disposed longitudinally in the lumen 206 of the tubular wall 200, towards the distal end portion 202 thereof. As such, the elongated control member 212 may or may not be slidably received through the aperture 900. Advantageously, when the elongated control member 212 is slidably received through the aperture 900, the aperture 900 enables speeding up the process of railing the elongated control member 212 along the length of the tubular wall 200, similarly to the use of a rapid exchange (RX) catheter system.

The elongated control member 212 may be made at least partially of a stiff material to provide sufficient structural rigidity to the elongated control member 212 for pushing the radially expandable member 210 outwardly from the lumen 206 of the tubular wall 200 without bending the elongated control member 212. In embodiment(s) where the elongated control member 212 is the hose members 404, 504, such stiff material may help preventing bending the hose members 404, 504 and possibly collapsing of the fluid passageways 408, 508 thereof upon applying a pushing force to the hose member 404, 504.

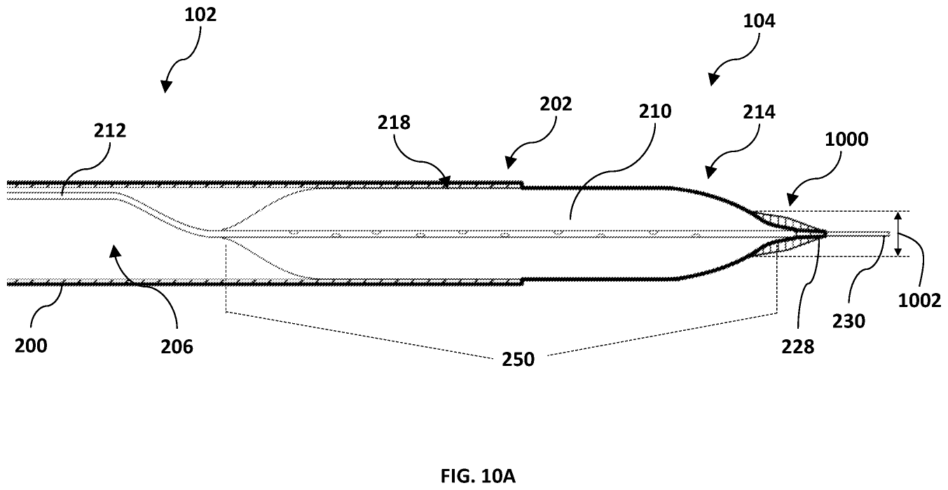

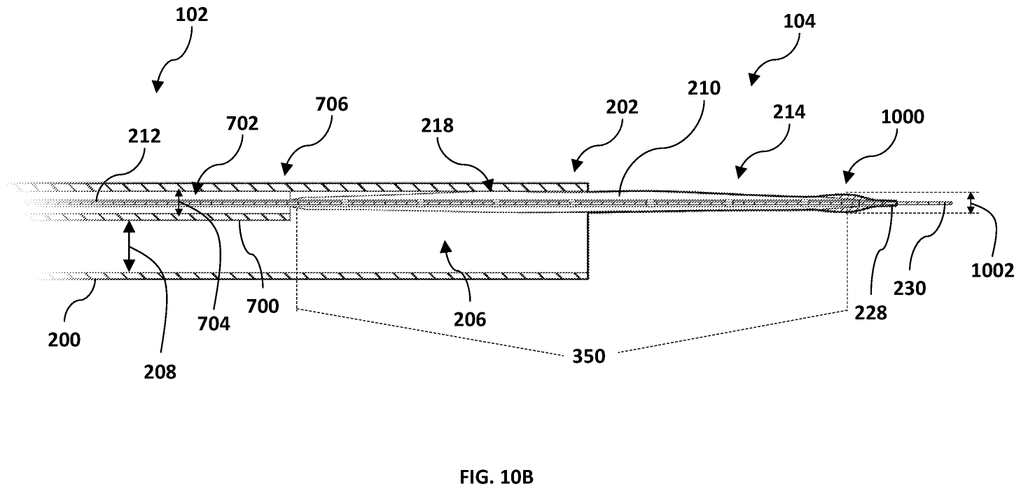

FIGS. 10A-B illustrate embodiments of the percutaneous assembly 100 for which the tapered end portion 214 of the radially expandable member 210 is provided with a reinforced distalmost narrower end 1000 to facilitate introduction of the percutaneous assembly 100 inside a body conduit of a subject. The reinforced distalmost narrower end 1000 has a fixed outer diameter 1002 which, along with the tapered shape of the reinforced distalmost narrower end 1000, remains constant regardless of whether the radially expandable member 210 is in the expanded configuration 250 (FIG. 10A) or the contracted configurations 350 (FIG. 10B). In embodiments where the tubular wall 200 is provided with a secondary tubular wall 700, as illustrated in FIG. 10B, the reinforced distalmost narrower end 1000 may be inserted in the secondary lumen 702 since the fixed outer diameter 1002 thereof is smaller than the secondary inner diameter 704 of the secondary lumen 702 defined by the secondary tubular wall.

Examples of materials for the reinforced distalmost narrower end 216 include non-deformable materials and/or materials such as biocompatible polymers like PTFE, TPE, polyethylene, etc.

Advantageously, a radially expandable member 210 provided with a reinforced distalmost narrower end 216 enables applying more force to the percutaneous assembly 100 during introduction into body conduit (as compared to a radially expandable member 210 without a reinforced distalmost narrower end 216) without causing structural deformation to the radially expandable member 210 that can compromise the structural integrity thereof.

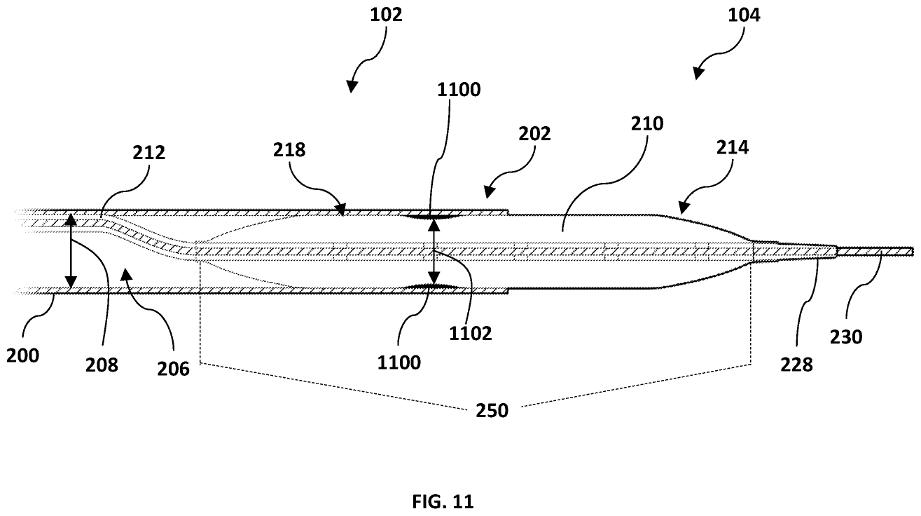

FIG. 11 illustrates an embodiment of the percutaneous assembly 100 for which the distal end portion 202 of the tubular wall 200 of the percutaneous sheath 102 is provided with an intraluminal portion 1100 that prevents displacement of the radially expandable member 210, when adjusted in the expanded configuration 250, in the lumen 206 of tubular wall 200. Particularly, the intraluminal portion 1100 has an inner diameter 1102 that is smaller than the expanded outer diameter 226 of the insertable end portion 218 of the radially expandable member 210 such that the insertable end portion 218 is immobilized in the lumen 206, preventing inward displacement of the radially expandable member 210 during introduction of the percutaneous assembly 100 into a body conduit of a subject.

In an embodiment, the insertable end portion 218 may be provided with a corresponding circumferential groove (not shown) sized and shaped to engage the intraluminal portion 1000 via an interference fit. In this case, for example, the radially expandable member 210 may be required to be adjusted in the contracted configuration 350 in order to be removed from the lumen 206 of the tubular wall 200.

The intraluminal portion 1100 of the tubular wall 200 may be made of a material that has a high friction coefficient with the insertable end portion 218 of the radially expandable member 210, such as biocompatible elastomer, providing a frictional engagement between the insertable end portion of the radially expandable member 210 and the intraluminal portion 1100 of the tubular wall 200.

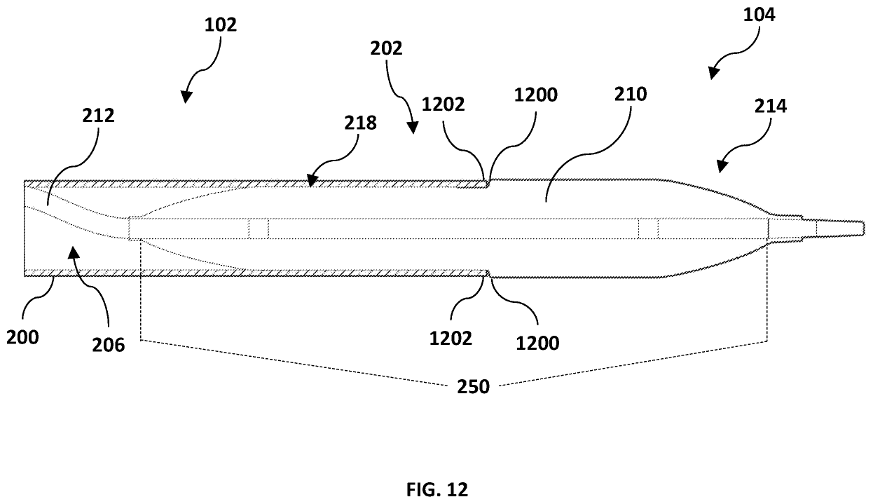

FIG. 12 illustrates an embodiment of the percutaneous assembly 100 for which the radially expandable member 210 of the percutaneous dilator tip 104 is provided with an abutment wall 1200 running at least partially along the circumference thereof. The abutment wall 1200, if present, delimits the tapered end portion 214 from the insertable end portion 218 of the radially expandable member 210. When the radially expandable member 210 is in the expanded configuration 250 and inserted in the lumen 206 of the tubular wall 200, the abutment wall 1200 abuts against a distalmost end 1202 of the distal end portion 202 of the tubular wall 200 to prevent inward displacement of the radially expandable member 210 during introduction of the percutaneous assembly 100 into a body conduit of a subject.

In embodiments, the attachment point 800 may be located between the abutment wall 1200 of the radially expandable member 210 and the distalmost end 1202 of the distal end portion 202 of the tubular wall 200 (not shown).

The skilled addressee will appreciate that the percutaneous sheath 102 may have various sizes and shapes, according to embodiments. For example, the percutaneous sheath 102 may have a circular, an oblong, or any other suitable transversal cross-section shape.

Generally speaking, the tubular wall 200 has a length about between 10 cm and 15 cm an outer diameter about between 5 French and 36 French and an inner diameter about between 4 French and 35 French. Exemplary material(s) from which the tubular wall 200 is made include biocompatible polymers like PTFE, TPE, and polyethylene.

It will further be appreciated that the percutaneous dilator tip 104, including the radially expandable member 210 and the elongated control member 212, may have various sizes and shapes, according to embodiments. For example, the radially expandable member 210 may have a circular, an oblong, or any other suitable transversal cross-section shape. The tapered end portion 214 of the radially expandable member 210 may be of a tapered shape, a rounded shape, a progressively narrowing shape, or any other shape suitable for introducing the percutaneous assembly 100 inside a body conduit. Furthermore, it is contemplated that the radially expandable member 210 may be non-inflatable and non-deflatable. For example, the radially expandable member 210 may be provided, for example, with one or more flexible or bendable element(s) that is or are biased towards a linear shape when the radially expandable member 210 is adjusted in a contracted configuration 350. Action of the elongated control member 210 to the radially expandable member 210 causes the flexible or bendable element(s) to bend so as the radially expandable member 210 is gradually adjusted in the expanded configuration 250 as the flexible or bendable element(s) bend(s). Alternatively, the radially expandable member 210 may be configured to self-contract when moved outwardly from the lumen 206 of the tubular wall 200 by the elongated control member 212 and be configured to self-expand when moved inwardly to the lumen 206 of the tubular wall 200 by the elongated control member 212. Other structures and/or mechanisms enabling the radially expandable member 210 to be adjusted in the expanded and contracted configurations 250, 350 under the action of the elongated control member 212 are possible and will be readily apparent to skilled address.

Generally speaking, the percutaneous dilator tip 104 has a length about between 10 cm and 300 cm, and an outer diameter about between 5 French and 36 French. The radially expandable member 210 has a length about between 1 cm and 10 cm, and an outer diameter about between 3 French and 36 French. The elongated control member 212 has a length about between 10 cm and 300 cm, and an outer diameter about between 1 French and 10 French. Exemplary material(s) from which the radially expandable member 210 is made include biocompatible polymers like PTFE, TPE, and polyethylene. Exemplary material(s) from which the elongated control member 212 is made include biocompatible polymer like PTFE, TPE, and polyethylene.

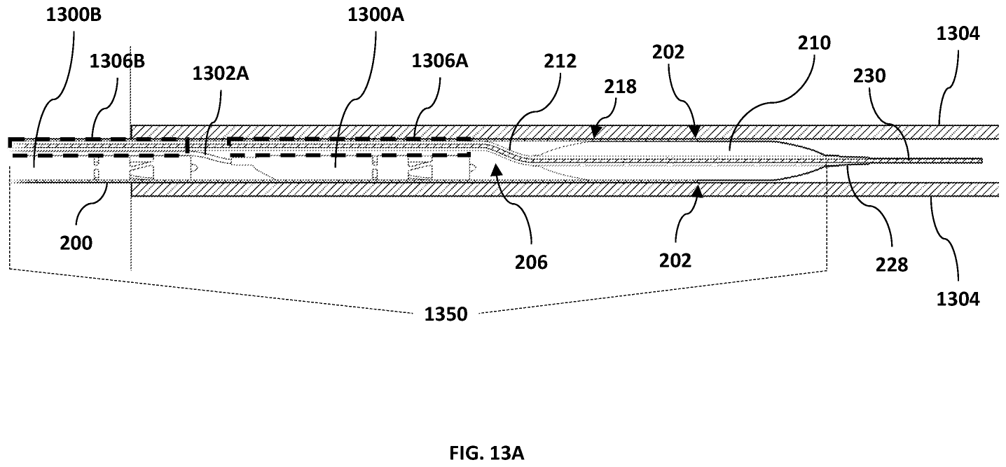

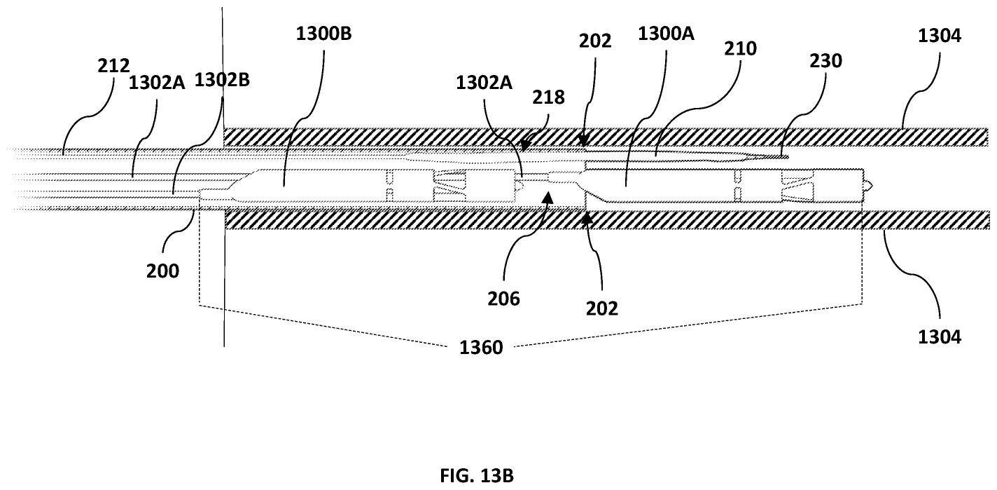

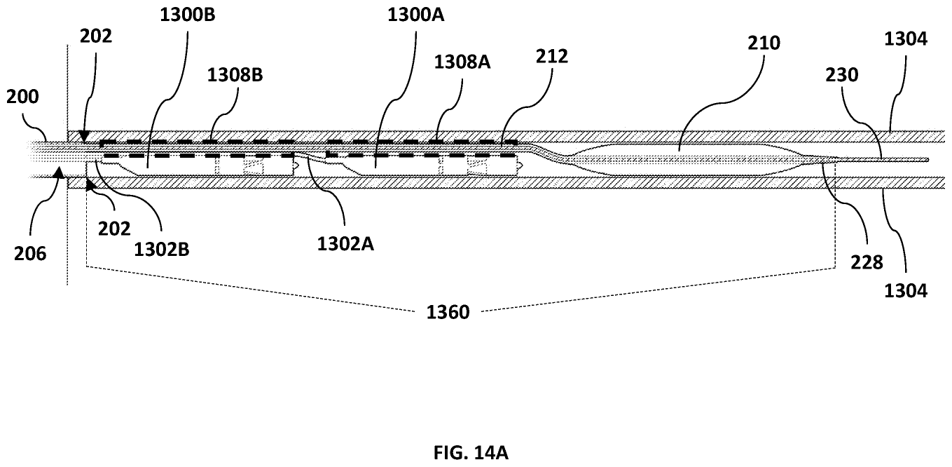

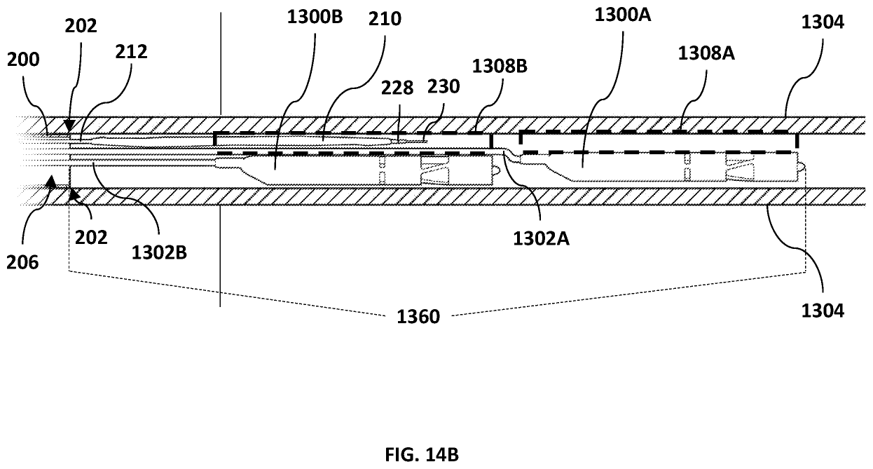

FIGS. 13A-B and 14A-B illustrate various embodiments of the percutaneous assembly 100 shown in use with at least one medical device, for example a first medical device 1300A provided with a first control wire 1302A connected thereto and a second medical device 1300B provided with a first control wire 1302B connected thereto. In these embodiments, the percutaneous assembly 100 may be used in various introduction arrangements for introducing the percutaneous assembly 100 containing the first and second medical devices 1300A and 1300B inside a body conduit 1304, and in various deployment arrangements for delivering and/or implanting the first and second medical devices 1300A and 1300B to a delivery and/or an implantation site(s) of a body conduit 1304.

As described hereinbefore for the elongated control member 212, the first and second control wires 1302A, 1302B serve not only to control or to power the first and second medical devices 1300A, 1300B (e.g., by electrically powering a heart pump(s)), but also serve to move longitudinally outwardly and inwardly the first and second medical devices 1300A, 1300B relative to the lumen 206 of the tubular wall 200. Particularly, the first and second medical devices 1300A, 1300B are moved outside of the lumen 206 of the tubular wall 200 by applying a pushing force to slide longitudinally the first and second control wire 1302A, 1302B outwardly in direction of the distal end portion 202 of the tubular wall 200. On the other hand, the first and second medical devices 1300A, 1300B are inserted in the lumen 206 of the tubular wall 200 by applying a pulling force to slide longitudinally the first and second control wires 1302A, 1302B inwardly in direction of the proximal end portion 204 of the tubular wall 200. For example, an operator may apply the pushing force to a proximal end portion (not shown) of the first and second control wires 1302A, 1302B to move outwardly the first and second medical devices 1300A, 1300B from the percutaneous sheath 102, and may apply a pulling force to the proximal end portion of the first and second control wires 1302A, 1302B to move inwardly the first and second medical devices 1300A, 1300B towards the percutaneous sheath 102.

In an embodiment, each of the first and second medical devices 1300A, 1300B may be moved outwardly and/or inwardly relative to the percutaneous sheath 102 independently and consecutively relative to one another. For example, a first heart pump may first be deployed into a blood vessel, followed by the deployment of a second blood pump.

In another embodiment, all of the first and second medical devices 1300A, 1300B may be moved outwardly and/or inwardly relative to the percutaneous sheath 102 simultaneously. For example, a first and a second heart pumps may be both deployed into a blood vessel at the same time.

FIG. 13A illustrates the percutaneous assembly 100 in an introduction arrangement 1350 and introduced inside a body conduit 1304, according to an embodiment. In the first introduction arrangement 1350, the first and second medical devices 1300A, 1300B are disposed in series inside the lumen 206 of the tubular wall 200, which may either be removably engaged or fixedly attached to the radially expandable member 210. Particularly, the first medical device 1300A is disposed towards the distal end portion 202 of the tubular wall 200, behind the radially expandable member 210 adjusted in the expanded configuration 250. The second medical device 1300B is disposed towards the proximal end portion 204 of the tubular wall 200, behind the first medical device 1300A. So disposed, each of the first and second medical devices 1300A, 1300B defines with the tubular wall 200 corresponding first and second inner sheath spaces 1306A and 1306B therebetween (the first and second inner sheath spaces 1306A, 1306B are indicated by bold dotted line rectangles in FIG. 13A). In the lumen 206 of the tubular wall 200, the first and second control wires 1302A and 1302B are disposed longitudinally in the lumen 206 of the tubular wall 200 and are positioned eccentrically relative to the lumen 206 thereof. The first control wire 1302A of the first medical device 1300A passes through the second inner sheath space 1306B, and the elongated control member 212 of the percutaneous dilator tip 104 passes through the first and second inner sheath spaces 1306A and 1306B.

FIG. 13B illustrates the percutaneous assembly 100 in a first deployment arrangement 1360 and introduced inside the body conduit 1304 for deploying at least one of the first and second medical devices 1300A, 1300B therein, according to an embodiment. In the first deployment arrangement 1360, the radially expandable member 210, which may either be removably engaged or fixedly attached to tubular wall 200, is adjusted in the contracted configuration 350 to provide at the distal end portion 202 of the tubular wall 200 the intraluminal clearance that is required for the first and second medical devices 1300A, 1300B to be moved outside of the lumen 206. As illustrated, the first medical device 1300A is moved outside of the lumen 206 of the tubular wall 200 to be deployed inside the body conduit 1304. Still contained in the lumen 206 of the tubular wall 200, the second medical device 1300B is partially moved towards the end portion 202 of the tubular wall 200. After having been moved outside of the lumen 206 of the tubular wall 200, the second medical device 1300B is deployed inside the body conduit 1304 between the tubular wall 200 and the first medical device 1300A, which is disposed further distally away from the tubular wall 200 (not shown).

As illustrated in FIG. 13B, where the radially expandable member 210 is adjusted in the contracted configuration 350 and is removably engaged to the tubular wall 200, the radially expandable member 210 may remain in place in the lumen 206 of the tubular 200 while the first and second medical devices 1300A, 1300B are moved outwardly from the tubular wall 200. Alternatively, the radially expandable member 210 may either be fully retracted in the lumen 206 of the tubular wall 200 or in the secondary lumen 702 of the secondary tubular wall 700. Particularly, the reinforced distalmost narrower end 216 of the radially expandable member 210 may also be fully retracted in the lumen 206 of the tubular wall 200 and in the secondary lumen 702 of the secondary tubular wall 700 since the fixed outer diameter 1002 of the reinforced distalmost narrower end 216 is smaller than the inner diameter 208 of the lumen 206 and the secondary inner diameter 704 of the secondary lumen 702, respectively.

FIGS. 14A-B illustrate the percutaneous assembly 100 in a second deployment arrangement 1362 and introduced inside the body conduit 1304 for deploying at least one of the first and second medical devices 1300A, 1300B therein, according to an embodiment. In the second deployment arrangement 1362, as illustrated in FIG. 14A, the radially expandable member 210 is removably engaged to the tubular wall 200 and is moved away from and disposed further distally of the distal end portion 202 thereof. As such, the radially expandable member 210 may be adjusted in either of the expanded configuration 250 (as shown in FIG. 14A) or the contracted configuration 350. The first and second medical devices 1300A, 1300B are moved outside of the lumen 206 of the tubular wall 200 and are deployed inside the body conduit 1304 between the tubular wall 200 and the radially expandable member 210. So deployed, each of the first and second medical devices 1300A, 1300B defines with the body conduit 1304 corresponding first and second inner body conduit spaces 1308A and 1308B therebetween (the first and second inner body conduit spaces 1308A, 1308B are indicated by bold dotted line rectangles in FIG. 14A-B). Inside the body conduit 1304, the first control wire 1302A of the first medical device 1300A passes through the second inner body conduit space 1308B, and the elongated control member 212 of the percutaneous dilator tip 104 passes through the first and second inner body conduit spaces 1308A and 1308B. Because of the disposition of the first and second medical devices 1300A, 1300B between the tubular wall 200 and the radially expandable member 210, the radially expandable member 210 is required to be adjusted in the contracted configuration 350 (as shown in FIG. 14B; if not already so adjusted previously (see below)) in order to be moved back towards the tubular wall 200 and to be passed through the first and second inner body conduit spaces 1308A, 1308B.

In the embodiments illustrated in FIGS. 13A-B and 14A-B, the at least one medical device may include a heart pump (also called pumping unit, functional unit or pumping module) that is operatively coupled to a control wire for electrically powering the heart pump.

Still in the embodiments illustrated in FIGS. 13A-B and 14A-B, the at least one medical device may include a plurality of modular heart pumps, each operatively coupled to a corresponding control wire for electrically powering the modular heart pump, that are assemblable together into a modular pump assembly (also called modular pump, modular functional unit, modular pumping unit or modular pumping module; not shown). For example, the at least one medical device may be three modular heart pumps.

Advantageously, the eccentric positioning of the elongated control member 212 relative to the lumen 206 of the tubular wall 200 in addition to the adjustability of the radially expandable member 210 in the expanded and contracted configurations 250, 350 enable medical device(s) that do not have an opening where the elongated control member 212 may pass therethrough (e.g., as it is the case for at least some of the prosthetic aortic valves, stents, endovascular stent grafts, and expandable filters known in the art) to be contained inside the percutaneous sheath 102 to be deployed inside a body conduit of a subject. Indeed, the positioning of the elongated control member 212 eccentrically relative to the lumen 206 of the tubular wall 210 creates the clearance required inside the tubular wall 200 for the medical device(s) to be contained therein without physically interfering with the elongated control member 212. After deployment of the medical device(s) inside a body conduit of a subject, the radially expandable member 210 is adjusted in the contracted configuration 350 so that it can be moved back towards the percutaneous sheath 102 and the percutaneous assembly 100 may be removed from the body conduit.

There are also disclosed kits, in accordance with a second aspect of the present disclosure. In an embodiment, a kit is provided with the percutaneous assembly 100 having the radially expandable member 210 of the percutaneous dilator tip 104 removably engageable to the tubular wall of the percutaneous sheath 102. In another embodiment, a kit is provided with the percutaneous assembly 100 having the radially expandable member 210 of the percutaneous dilator tip 104 removably engageable to the tubular wall of the percutaneous sheath 102, and at least one medical device containable inside the percutaneous assembly 100. In still another embodiment, a kit is provided with the percutaneous assembly 100 having the radially expandable member 210 of the percutaneous dilator tip 104 fixedly engaged to the tubular wall of the percutaneous sheath 102, and at least one medical device containable inside the percutaneous assembly 100.

Various methods of deploying at least one medical device into a body conduit of a subject through a body conduit access using the percutaneous assembly 100 is disclosed, in accordance with a third aspect of the present disclosure.

In an embodiment, the at least one medical device deployed includes at least one heart pump (also called pumping unit, functional unit or pumping module).

In another embodiment, the at least one medical device deployed includes at least one modular heart pumps that may be assembled together into a modular pump assembly (also called modular pump, modular functional unit, modular pumping unit or modular pumping module; not shown). For example, percutaneous assembly 100 may be provided with three modular heart pumps.

Still in another embodiment, the body conduit includes a delivery site where the at least one medical device is deployed (i.e., delivered) thereto and/or an implantation site where the at least one medical device is deployed (i.e., implanted) thereto.

Yet in another embodiment, the body conduits into which the at least one medical device is deployed includes a blood vessel.

It will be appreciated that a radially expandable member, which is similar to the radially expandable member 210 and may include an inflatable structure, and an elongated control member, which is similar to the elongated control member 212 and may include a hose for inflating the inflatable structure, may be provided to any medical devices of interest. Such medical devices of interest include any medical devices capable of structurally integrating a radially expandable member and an elongated control member, and is so sized and shaped to be used in a transcatheter procedure, with the necessary change(s), appreciable to the skilled addressee, having been made. In this case, the medical device of interest is positioned to the distal end portion of a sheath, for example one similar to the percutaneous sheath 102, such that the sheath containing the medical device of interest may be introduced into a body conduit of a subject, like the vasculature, for delivery and/or implantation of the medical device of interest. The presence of the radially expandable member or inflatable structure in the expanded configuration on the periphery of the medical device of interest conform to the distal end portion of the sheath, as described hereinbefore, and thus provides one or more or the advantages described hereinbefore.

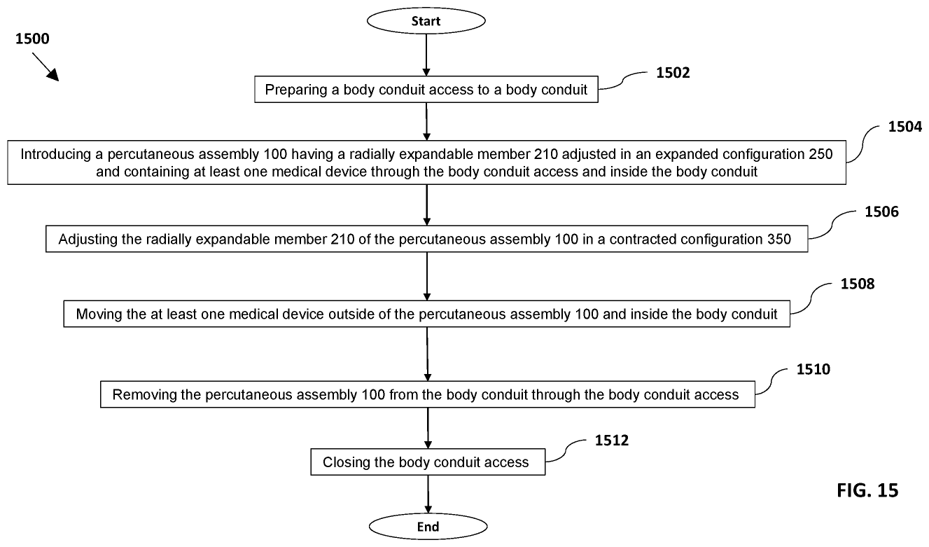

FIG. 15 schematizes a first method 1500 of deploying at least one medical device to a body conduit, according to an embodiment. The first method 1500 includes:

Inserting at least one medical device into the lumen 206 of the tubular wall 200 of the percutaneous sheath 102 via the distal end portion 202 or the proximal end portion 204. If the medical device is inserted in the lumen 206 via the distal end portion 202 of the tubular wall 200, inserting the radially expandable member 210 in the lumen 206 of the tubular wall 200 at the distal end portion 202 thereof. If the radially expandable member 210 is in the contracted configuration 350, adjusting the radially expandable member 210 in the expanded configuration 250.

Preparing a body conduit access to a body conduit where the percutaneous assembly 100 is to be introduced, at step 1502. In an embodiment, the body conduit access is a vascular body conduit access, such as a transfemoral, a transaxillary, and a radial artery body conduit access. It is contemplated that preparing a body conduit access includes preparing of a natural body conduit access, such as esophageal, tracheal, urethral, rectal, or transcutaneous access, which may for example requires anesthesia or any other medical or surgical preparation.

Optionally, introducing a guidewire through the body conduit access of the body conduit. Still optionally, guiding the guidewire up to a delivery site and/or an implantation site of the body conduit. Yet optionally, railing a catheter of the percutaneous assembly 100 over the guidewire.

Introducing the percutaneous assembly 100 with the radially expandable member 210 first (adjusted in the expanded configuration 250) through the body conduit access and inside the body conduit, at step 1504 (FIG. 13A illustrates an embodiment of the percutaneous assembly 100 in the introduction arrangement 1350 that may be used for this step).

Advancing the percutaneous assembly 100 along the body conduit to the delivery site and/or an implantation site thereof, if the delivery site and/or an implantation site of the body conduit where the at least one medical device is to be implemented is distant from the body conduit access. Optionally, using the guidewire for advancing the percutaneous assembly 100.

Adjusting the radially expandable member 210 in the contracted configuration 350 with the elongated control member 212, at step 1506.

Optionally, in embodiments for which the radially expandable member 210 is removably engageable, such as removably insertable, to the tubular wall 200, retracting the radially expandable member 210 adjusted in the contracted configuration 350 towards the proximal end portion 204 of the tubular wall 200 such that the radially expandable member 210 is fully inserted in the lumen 206 of the tubular wall 200, for example by an operator pulling on the proximal end portion 222 of the elongated control member 212. Still optionally, alternatively moving the radially expandable member 210 away from the distal end portion 202 of the tubular wall 200, for example by an operator pushing on the proximal end portion 222 of the elongated control member 212.

Moving the at the least one medical device outside of the lumen 206 of the tubular wall 200 and inside the body conduit at the delivery site and/or an implantation site thereof, at step 1508, for example by an operator pushing on the proximal end portion of the control wire 1302A, B (FIGS. 13B and 14A-B illustrate embodiments of the percutaneous assembly 100 in the first and second deployment arrangements 1360, 1362 that may be used for this step).

Removing the percutaneous assembly 100 from the body conduit through the body conduit access, at step 1510.

Closing the body conduit access of the body conduit, at step 1512. In an embodiment, the body conduit access may be closed with at least one of the control wires 1302A, 1302B protruding therefrom.

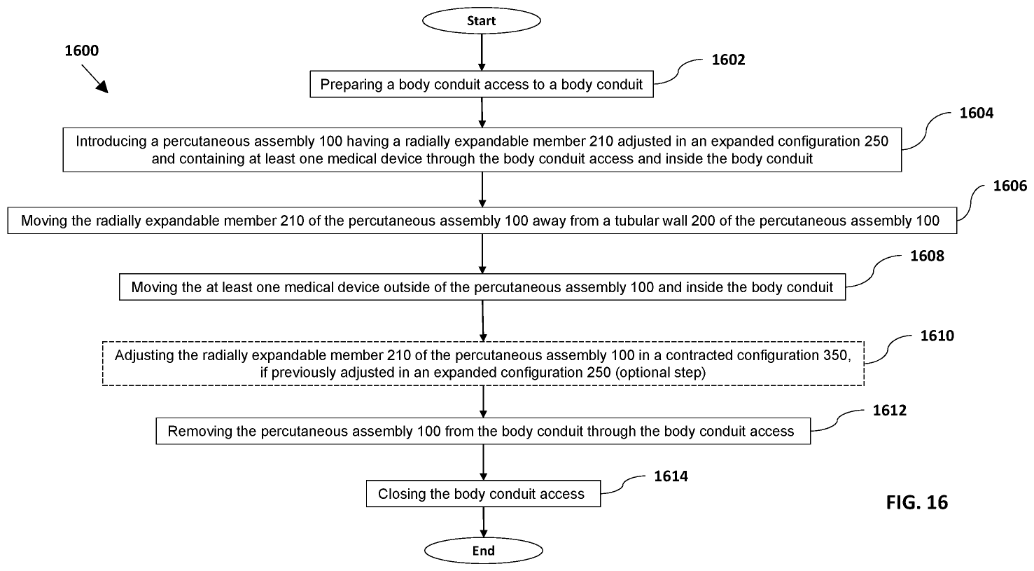

FIG. 16 schematizes a second method 1600 of deploying at least one medical device to a body conduit, according to another embodiment. The second method 1600 includes:

Inserting at least one medical device into the lumen 206 of the tubular wall 200 of the percutaneous sheath 102 via the distal end portion 202 or the proximal end portion 204. If the medical device is inserted in the lumen 206 via the distal end portion 202 of the tubular wall 200, inserting the radially expandable member 210 in the lumen 206 of the tubular wall 200 at the distal end portion 202 thereof. If the radially expandable member 210 is in the contracted configuration 350, adjusting the radially expandable member 210 in the expanded configuration 250.

Preparing a body conduit access to a body conduit where the percutaneous assembly 100 is to be introduced, at step 1602. In an embodiment, the body conduit access is a vascular body conduit access, such as a transfemoral, a transaxillary, and a radial artery body conduit access. It is contemplated that preparing a body conduit access includes preparing of a natural body conduit access, such as esophageal, tracheal, urethral, rectal, or transcutaneous access, which may for example requires anesthesia or any other medical or surgical preparation.

Optionally, introducing a guidewire through the body conduit access of the body conduit. Still optionally, guiding the guidewire up to a delivery site and/or an implantation site of the body conduit. Yet optionally, railing a catheter of the percutaneous assembly 100 over the guidewire.

Introducing the percutaneous assembly 100 with the radially expandable member 210 first (adjusted in the expanded configuration 250) through the body conduit access and inside the body conduit, at step 1604 (FIG. 13A illustrates an embodiment of the percutaneous assembly 100 in the introduction arrangement 1350 that may be used for this step).

Advancing the percutaneous assembly 100 along the body conduit to the delivery site and/or an implantation site thereof, if the delivery site and/or an implantation site of the body conduit where the at least one medical device is to be implemented is distant from the body conduit access. Optionally, using the guidewire for advancing the percutaneous assembly 100.

Moving the radially expandable member 210 adjusted in the expanded configuration 250 or in the contracted configuration 350 distally away from the distal end portion 202 of the tubular wall 200 of the percutaneous sheath 102, at step 1606, for example by an operator pushing on the proximal end portion 222 of the elongated control member 212.

Moving the at the least one medical device outside of the lumen 206 of the tubular wall 200 and inside the body conduit at the delivery site and/or an implantation site thereof, at step 1608, for example by an operator pushing on the proximal end portion of the control wire 1302A,B (FIGS. 13B and 14A-B illustrate embodiments of the percutaneous assembly 100 in the first and second deployment arrangements 1360, 1362 that may be used for this step).

Adjusting the radially expandable member 210 in the contracted configuration 350, at step 1610, if the radially expandable member 210 in the expanded configuration 250 has been moved away from the distal end portion 202 of the tubular wall 20 (this step is shown with a dotted line in FIG. 16 to indicate that this step is optional depending on which of the expanded and contracted configurations 250 and 350 the radially expandable member 210 is adjusted to). As such, the adjustment in the contracted configuration 250 allows the radially expandable member 210 is to be sized and shaped to be able to pass through at least one body conduit intraluminal space defined between the body of at least one medical device deployed in the body conduit and the inner wall (which correspond to the intima in case of blood vessel) of the body conduit (such passing through the body conduit intraluminal space(s) would not be necessarily feasible due to physical hinderance when the radially expandable member 210 is maintained in the expanded configuration 250).

Optionally, adjusting the radially expandable member 210 in the expanded configuration 250 before removing the percutaneous assembly 100 from the body conduit.

Removing the percutaneous assembly 100 from the body conduit through the body conduit access, at step 1612.

Closing the body conduit access of the body conduit, at step 1614. In an embodiment, the body conduit access may be closed with at least one of the control wires 1302A, 1302B protruding therefrom.

1. A percutaneous assembly, comprising:
a percutaneous sheath having a tubular wall extending to a distal end portion thereof, the tubular wall defining a lumen having an inner diameter;
a percutaneous dilator tip including a radially expandable member and an elongated control member;
the radially expandable member having an expandable end portion removably engageable to the distal end portion of the tubular wall, the radially expandable member being adjustable in an expanded configuration in which the expandable end portion is configured to conform to the distal end portion of the tubular wall when engaged thereto, and a contracted configuration in which the expandable end portion has a contracted outer diameter that is smaller than the inner diameter of the lumen of the tubular wall; and
the elongated control member being operatively coupled to the radially expandable member for adjusting the radially expandable member in at least one of the expanded configuration and the contracted configuration, the elongated control member further being longitudinally movable relative to the tubular wall when the expandable end portion of the radially expandable member is in the contracted configuration, the elongated control member further being eccentrically positioned relative to the lumen of the tubular wall.

2. The percutaneous assembly according to claim 1, wherein the expandable end portion of the radially expandable member is removably engageable by being removably insertable in the lumen of the tubular wall at the distal end portion thereof.

3. The percutaneous assembly according to claim 2, wherein the distal end portion of the tubular wall is configured for fitting snuggly with the expandable end portion of the radially expandable member adjusted in the expanded configuration when inserted in the lumen of the tubular wall.

4. The percutaneous assembly according to claim 2, wherein the distal end portion of the tubular wall includes an intraluminal portion having an inner diameter that is smaller than the expanded outer diameter of the expandable end portion of the radially expandable member when inserted in the lumen of the tubular wall.

5. The percutaneous assembly according to claim 1, wherein the expandable end portion of the radially expandable member adjusted in the expanded configuration has an expanded outer diameter that is substantially equal to the inner diameter of the lumen of the tubular wall when the expandable end portion is connected thereto.

6. The percutaneous assembly according to claim 1, wherein the elongated control member is longitudinally slidably receivable in the lumen of the tubular wall.

7. The percutaneous assembly according to claim 1, wherein the percutaneous sheath includes a secondary tubular wall extending at least partially along the tubular wall and defining a secondary lumen, the elongated control member being longitudinally slidably receivable in the secondary lumen.

8. The percutaneous assembly according to claim 1, further comprising a catheter for guiding the percutaneous assembly over a guidewire along a body conduit of a subject, the catheter being positioned eccentrically relative to the lumen of the tubular wall along at least a portion thereof.

9. The percutaneous assembly according to claim 1, wherein the radially expandable member includes a tapered end portion longitudinally opposed to the expandable end portion the tapered end portion of the radially expandable member comprising a reinforced distalmost narrower end.

10. The percutaneous assembly according to claim 1, wherein the radially expandable member is a balloon member of a balloon catheter.

11. The percutaneous assembly according to claim 10, wherein the elongated control member is a hose member of the balloon catheter and is configured for adjusting the balloon member in the expanded configuration by inflation and in the contracted configuration by deflation.

12. The percutaneous assembly according to claim 1, further comprising at least one medical device contained in the lumen of the tubular wall, the at least one medical device being movable longitudinally outwardly from the lumen, at the distal end portion of the tubular wall, when the expandable end portion of the radially expandable member is removed from the distal end portion of the tubular wall.

13. The percutaneous assembly according to claim 12, wherein the at least one medical device and the tubular wall together define a first inner sheath space therebetween where the elongated control member passes therethrough.

14. The percutaneous assembly according to claim 13, wherein the contracted outer diameter of the radially expandable member is smaller than the inner sheath space.

15. The percutaneous assembly according to claim 12, wherein the at least one medical device is free of an opening through which the elongated control member can be passed.

16. The percutaneous assembly according to claim 12, wherein the at least one medical device includes a first medical device and a second medical device disposed proximally to the first medical device, the first medical device having a first control wire connected thereto, the first control wire disposed longitudinally in the lumen of the tubular wall and positioned eccentrically relative to the lumen, the second medical device and the tubular wall together defining a second inner sheath space therebetween where the first control wire passes therethrough.

17. A percutaneous assembly, comprising:
a percutaneous sheath having a tubular wall extending to a distal end portion thereof, the tubular wall having a circumference and defining a lumen having an inner diameter;
a percutaneous dilator tip including a radially expandable member and an elongated control member;
the radially expandable member having an expandable end portion fixedly attached on a portion of the circumference of the tubular wall at the distal end portion thereof, the radially expandable member being adjustable in an expanded configuration in which the expandable end portion is configured to conform to the distal end portion of the tubular wall, and a contracted configuration in which the expandable end portion has a contracted outer diameter that is smaller than the inner diameter of the lumen of the tubular wall; and
the elongated control member being operatively coupled to the radially expandable member for adjusting the radially expandable member in at least one of the expanded configuration and the contracted configuration, the elongated control member further being longitudinally disposed relative to the tubular wall and eccentrically positioned relative to the lumen thereof.

18. The percutaneous assembly according to claim 17, wherein the expandable end portion of the radially expandable member is received in the lumen of the tubular wall at the distal end portion thereof.

19. The percutaneous assembly according to claim 17, wherein the elongated control is structurally integrated to the tubular wall.

20. The percutaneous assembly according to claim 17, further comprising a catheter for guiding the percutaneous assembly over a guidewire along a body conduit of a subject, the catheter being positioned eccentrically relative to the lumen of the tubular wall along at least a portion thereof.

21. The percutaneous assembly according to claim 20, wherein the catheter is structurally integrated to the tubular wall.

22. The percutaneous assembly according to claim 17, wherein the radially expandable member is a balloon member of a balloon catheter.

23. The percutaneous assembly according to claim 22, wherein the elongated control member is a hose member of the balloon catheter and is configured for adjusting the balloon member in the expanded configuration by inflation and in the contracted configuration by deflation.

24. A kit, comprising:
c) a percutaneous sheath having a tubular wall extending to a distal end portion thereof, the tubular wall defining a lumen having an inner diameter;
d) a percutaneous dilator tip including a radially expandable member and an elongated control member;
the radially expandable member having an expandable end portion removably engageable to the distal end portion of the tubular wall, the radially expandable member being adjustable in an expanded configuration in which the expandable end portion is configured to conform to the distal end portion of the tubular wall when engaged thereto, and a contracted configuration in which the expandable end portion has a contracted outer diameter that is smaller than the inner diameter of the lumen of the tubular wall; and
the elongated control member being operatively coupled to the radially expandable member for adjusting the radially expandable member in at least one of the expanded configuration and the contracted configuration, the elongated control member further being longitudinally movable relative to the tubular wall when the expandable end portion of the radially expandable member is in the contracted configuration, the elongated control member further being eccentrically positioned relative to the lumen of the tubular wall,
e) at least one medical device containable in the lumen of the tubular wall.

25. A method of deploying a medical device inside a body conduit of a subject through a body conduit access, the method comprising:
introducing a percutaneous assembly through the body conduit access inside the body conduit, the percutaneous assembly comprising a percutaneous sheath having a tubular wall extending to a distal end portion thereof and defining a lumen, and a percutaneous dilator tip having a radially expandable member disposed at the distal end portion of the tubular wall and adjusted in an expanded configuration, the tubular wall containing the medical device in the lumen thereof;
moving the radially expandable member distally away from the distal end portion of the tubular wall;
moving the medical device at least partially outside of the lumen of the tubular wall inside the body conduit;
adjusting the radially expandable member in a contracted configuration; and
removing the percutaneous assembly from the body conduit through the body conduit access.

26. The method according to claim 25, further comprising:
preparing a body conduit access to the body conduit.

27. The method according to claim 25, wherein said introducing includes advancing the percutaneous assembly inside the body conduit.

28. The method according to claim 25, wherein said introducing includes advancing the percutaneous assembly over a guidewire inside the body conduit.

29. The method according to claim 25, wherein said removing includes passing the radially expandable member adjusted in the contracted configuration through an inner body conduit space defined by the medical device and the body conduit.

30. The method according to claim 25, further comprising:
closing the body conduit access of the body conduit.
